(19) United States
(12) Patent Application Publication
(10) Pub. No.: US 2026/0256446 A1
(43) Pub. Date: Sep. 3, 2026

(54) A DEVICE TO ASSESS INTRACRANIAL PRESSURE (ICP) NONINVASIVELY

(71) Applicant: The Regents of the University of California, Oakland, CA (US)

(72) Inventors: Paul M. Vespa, Los Angeles, CA (US); Iheanacho Emeruwa, Los Angeles, CA (US); Gabriel L. Oland, Los Angeles, CA (US); Neil Parikh, Los Angeles, CA (US); Gil Herrnstadt, Los Angeles, CA (US)

(73) Assignee: The Regents of the University of California, Oakland, CA (US)

(21) Appl. No.: 19/163,656

(22) PCT Filed: Mar. 26, 2024
(86) PCT No.: PCT/US24/21450

§ 371 Date: Sep. 9, 2025

Related U.S. Application Data
(60) Provisional application No. 63/492,437, filed on Mar. 27, 2023.

(51) Int. Cl. A61B 8/08; A61B 8/00; A61B 8/10

(52) U.S. Cl. CPC A61B 8/0808; A61B 8/10; A61B 8/4218; A61B 8/4227; A61B 8/4416; A61B 8/5223; A61B 8/5292

(57) ABSTRACT

The disclosure provides systems, methods and materials useful for the noninvasive estimation of intracranial pressure. Embodiments of the invention include systems for estimating intracranial pressure in a subject which comprise a headset adapted to rest on the head and/or face of the subject and at least one sensing probe. In such systems, the sensing probe is coupled to the headset in an orientation such that the sensing probe can sense phenomena such as optic nerve diameter and/or optic nerve sheath diameter. In illustrative systems, the sensing probe is adapted to capture sensed parameters and the system then correlates one or more sensed parameters with intracranial pressure so as to estimate intracranial pressure in the subject noninvasively.

### CROSS REFERENCE TO RELATED APPLICATIONS

This application claims the benefit under 35 U.S.C. Section 119 (e) of co-pending and commonly-assigned U.S. Provisional Patent Application No. 63/492,437, filed Mar. 27, 2023, entitled “A DEVICE TO ASSESS INTRACRANIAL PRESSURE (ICP) NONINVASIVELY”, which application is incorporated by reference herein.

### TECHNICAL FIELD

The present invention is in the technical field of devices that estimate intracranial pressure (ICP). More specifically, the present invention is in the technical field of non-invasive or minimally invasive devices for estimating ICP.

### BACKGROUND OF THE INVENTION

ICP dysregulation is a life-threatening issue, particularly for the combined 3.3 million patients in the United States that annually suffer from cardiac arrest, stroke, intracranial hemorrhage, and traumatic brain injury. Elevations of ICP>20 mmHg can result in secondary brain injury that compounds the initial insult, resulting in tissue hypoxia, cerebral edema, brain herniation, and death. Currently there are no reliable methods for monitoring the absolute ICP without directly inserting a catheter through brain tissue, which carries the risks of infection (9%) and serious hemorrhage (1-2%). Additionally, many conditions may preclude invasive monitoring, such as intrinsic coagulopathy, antiplatelet or anticoagulant use, and high infection risk. Thus, ICP monitoring is being underutilized in often the most critically ill patients, leading to lack of brain-directed therapies.

Non-invasive surrogates for ICP monitoring have been investigated in the last two decades and include techniques like transcranial doppler (TCD), pupillometry, electroencephalography (EEG), and near-infrared spectroscopy (NIRS), although each have certain limitations. One well-validated method uses ultrasound to measure the optic nerve sheath diameter (ONSD), as the nerve sheath is contiguous with the subarachnoid space around the brain and swells with increasing ICP. Numerous studies have corroborated its correlation to ICP with median correlation r=0.61 (IQR 0.56-0.78) and more refined studies even tout area under the ROC curve as high as 0.94. However, this ROC curve data describes a binary outcome, i.e. indicating only whether ICP is “normal” or “elevated,” and therefore does not correlate with ICP across a wide range of values. Additionally, in conventional systems, the data is static in that ultrasound measurements are relatively burdensome to acquire and thus are only measured a few times a day, as compared to ICP, which is dynamic and fluctuates on a moment-to moment basis.

For the reasons noted above, there is a need in this technology for non-invasive devices and methods designed for continuously estimating a patient's ICP.

### SUMMARY OF THE INVENTION

Embodiments of the present invention provide non-invasive devices and associated methods for continuously estimating a patient's ICP. The instant disclosure describes hands-free methods of collecting continuous, trended ICP data. The data obtained by such embodiments of the invention is vastly more comprehensive than those generated by conventional methods, and therefore significantly more actionable to clinicians than current non-invasive methods.

In addition to stroke, intracranial hemorrhage, and traumatic brain injury, where embodiments of the invention can dramatically influence the speed of detection of ICP changes, embodiments of the invention also have enormous treatment implications for any patient with risk factors for elevated ICP, including central nervous system infections and neoplasms, hydrocephalus, encephalopathy, and hypertensive urgency or emergency. For example, embodiments of the invention can be used to screen patients in the emergency department or trauma bay that have a Glasgow Coma Scale <15, are altered or intoxicated, or have a neurologic deficit. In the operating room embodiments of thus system/device can be employed during any case known to elevate ICP, including those which involve cardiac bypass, abdominal or thoracic insufflation, or prone or Trendelenburg positioning. The ultimate benefit of the systems disclosed herein include a low-profile, non-invasive monitoring device that has high correlation with ICP, and provides continuous and actionable data to clinicians without increasing labor or alarm fatigue. Consequently, embodiments of the invention can be used to obtain information and provide results that can optimize clinical outcomes for patients while simultaneously providing increased cost-savings for both providers and payers by reducing complications and length of stay in the hospital.

Embodiments of the invention include, for example, systems and associated methods for estimating intracranial pressure in a subject. Typically these systems comprise a headset adapted to rest on the head and/or face of the subject; and at least one sensing probe; wherein the sensing probe is coupled to the headset and adapted to capture a plurality of sensed parameters from a plurality of axes; wherein the system correlates one or more sensed parameters with intracranial pressure so as to estimate intracranial pressure in the subject noninvasively, and optionally, continuously.

As noted above, embodiments of the present invention comprise a hardware device that allows for noninvasive recording of patient data. The hardware device is composed of a mountable headset to be left about or on a patient's head to continuously take automated measurements (or intermittently take measurements throughout the day, with the capability of turning on and off at pre-specified time points). The device incorporates a plurality of sensing modalities including, but not limited to, ultrasound, Doppler ultrasound, pupillometry, dynamometry, near infrared spectroscopy, acoustic tympanometry, optical coherence tomography, and electroencephalography. Any combination of the above-mentioned modalities, along with other patient data, may be used to enhance the accuracy of the ICP estimation with the device. The sensing modalities probes may be attached along the top, front and sides of the device frame. The sensing modalities may be adjustable along the frame of the mountable headset so as to acquire measurements from varying angles and of varying structures about the head. For example, an ultrasound sensing modality attached along the top frame of the device can have the ability to be moved along multiple axes in space, including axial, sagittal, coronal, and rotational axes. The measurements acquired with the device may be either static or dynamic over time.

The present invention includes further methods of utilizing a combination of sensing modalities to measure anatomical features and parameters related to the brain, eye, and optic nerve, so as to obtain an accurate estimation of ICP.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides a perspective view of a mountable headset device of the present invention with several measuring modalities connected at various possible locations;

FIG. 2 provides a perspective view of an embodiment of the mountable headset device of the present invention showing 3 modalities, including ultrasound mounted over the bilateral eyes, transcranial doppler ultrasound mounted over the temporal acoustic windows, and pupillometry mounted over the bilateral eyes;

FIG. 3 provides a perspective view of the mountable headset device of FIG. 1 showing a measuring modality having multiple degrees of freedom (DOF) of adjustment;

FIG. 4 provides afront view of the present invention showing a medium such as a liquid gel or a solid but deformable gelatinous material embedded in the mountable headset;

FIG. 5 provides afront view of a hollow structure design that allows a probe to move freely in multiple degrees of freedom while maintaining constant contact with the eyelid;

FIG. 6 provides a front view of a mountable headset device of the present invention, in the form of a headband, with a single measuring modality;

FIG. 7 provides atop view of a mountable headset device of the present invention, with curved probe located medially; a zoomed in top view of the curved probe is shown with pressure sensor attachment;

FIG. 8 is an assembly view of an iteration of the headframe that is strapped about the head and transducer(s) that rests on and makes contact with the face with multiple degrees of freedom;

FIG. 9 is an assembly view of an iteration of the headframe that rests on the bridge of the nose and bilateral ears and transducer(s) that rests against and makes contact with the face with multiple degrees of freedom;

FIG. 10 is an axial cross-sectional view of the structure of the globe and optic nerve identifying features of interest for the present invention;

FIG. 11 is an axial cross-sectional view of the structure of the globe and optic nerve structure undergoing displacement due to elevated ICP, and corresponding features of interest for the present invention;

FIG. 12 is an axial cross-sectional view of the structure of the globe and optic nerve undergoing distention due to elevated ICP, and corresponding features of interest for the present invention;

FIG. 13 shows a sequence of raw and binary images of the anatomical region of interest and features for motion tracking;

FIG. 14 shows a binary image overlayed over the respective raw image;

FIG. 15 shows the power spectral density of detected motion consisting of a perturbation signal frequency and the measured response fundamental frequency and higher harmonics;

FIG. 16 shows a spectral analysis of the ONSD and surrounding structure motions via pixel intensity. Identified pixels are highlighted;

FIG. 17 shows spectral analysis of translations in x and y directions of a selected area of pixels (similar to FIG. 16) estimated using intensity-based registration;

FIG. 18 shows comparison of ocular ultrasound recorded manually and with the device in this invention and subjected to similar spectral analysis,

FIG. 19 shows a diagram depicting the process for performing spectral analysis with ocular ultrasound recorded manually vs with the device in this invention;

FIG. 20 shows high frequency parameters extracted from spectral analysis in relation to the subject posture;

FIG. 21 shows errors between the heart rate extracted from the ultrasound signal (recorded manually or with the device in this invention) and a physical recording;

FIG. 22 shows an input image for the machine learning model, its manually labelled ground truth, and its output;

FIG. 23 shows a cross-section view of the optic nerve with multiple measurements, and a perspective view of the eye showing an axis between the probe and the optic nerve (i.e., probe-nerve axis);

FIG. 24 provides aside and top views of anatomic positioning of ultrasound probe angles;

FIG. 25 provides a block diagram representing a feedback control approach of the present invention;

FIG. 26 provides a block diagram representing an expanded feedback control approach of FIG. 18;

FIG. 27 provides a block diagram representing a feedback loop of the present invention implementation with a healthcare provider embedded in the loop;

FIG. 28 provides a block diagram representing a feedback loop of the present invention implementation with external equipment embedded in the loop;

FIG. 29 is an assembly view of an iteration of the headframe that rests on the bridge of the nose and bilateral ears and transducer(s) that rests against and makes contact with the face with multiple degrees of freedom;

FIG. 30 provides a composition of isometric, front, top and right assembly view of an iteration of the headframe that rests on the bridge of the nose and bilateral ears and transducer(s) that rests against and makes contact with the face with multiple degrees of freedom;

FIG. 31 provides photos and schematics showing how ocular ultrasound enables sensitive estimation of elevated ICP through measurement of the optic nerve sheath;

FIG. 32 provides photos showing an embodiment of the invention for the non-invasive, continuous ICP monitoring. In this embodiment, the wearable sits on the patient's nasal bridge and precisely holds an ocular ultrasound probe in place, with the probe secured at eyelid for measurement of one or both optic nerves. The device enables continuous measurements on sedated patients, or it can be used for individual measurements on non-sedated and awake patients;

FIG. 33 provides photos showing a number of embodiments of the invention that are designed for continuous, hands-free ocular ultrasound. A) shows the version 1 prototype headframe, B) shows active ultrasound data collection of the optic nerve with probe mounted in the headframe over the left upper eyelid, C/D) shows assembled headframe prototype version 2 with the ability to mount C) unilateral or D) bilateral probes, E) shows participant wearing a version 2 prototype;

FIG. 34 provides graphed data showing high frequency motion signals derived from ultrasound images may improve ICP estimation. Top panel shows heart rate data derived from both ultrasound and standard heart rate monitor in 6 healthy participants collected in upright (0°) and supine (90°) positions. Bottom panel shows three high frequency signals from image analysis for the same 6 participants. High heart rate correlates with increased HF2 and HF3 and decreased HF1. High frequency signals are also associated with change in position;

FIG. 35 provides an isometric view of an embodiment of the invention comprising a wearable headband device (top panel) and a side view of an embodiment of the invention comprising a wearable headband device (bottom panel);

FIG. 36 provides front, side, top, and isometric views of an embodiment of the invention comprising a wearable headband device; and

FIG. 37 provides an illustration of an embodiment of the invention comprising a wearable headband device with a standard ultrasound probe. The device attaches to the headband with a magnetic base which allows the user to position the mechanism anywhere on the metal sheet. In addition, the linear and two spherical joints enable probe positioning to capture the anatomy of the eye and optic nerve. A single knob enables to lock and release the spherical joints. When used to capture ultrasound imaging from patients in supine and 30° positions, the gravitational force facilitates consistent contact over the eye without a linear joint lock. Alternative embodiments include a linear joint lock.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

Elevated intracranial pressure (ICP) is associated with significant morbidity and mortality. Elevated ICP, or intra-cranial hypertension, often results from traumatic brain injuries (TBIs), cardiac arrest, and ischemic and hemorrhagic stroke, for which there are an estimated 3.6 million combined patients in the U.S. each year (92% are TBI and stroke alone). There were over 64,000 TBI related deaths in the U.S. in 2020 and more than 223,000 TBI-related hospitalizations. Moderate and severe TBIs are associated with poor long-term outcomes where approximately 30% of patients showed worse symptoms following treatment and 22% had died after 5 years. While much of the morbidity and mortality of TBI is associated with the primary brain damage event, secondary brain damage (often from cerebral edema or ischemia) can present hours to days after the initial event. The Brain Trauma Foundation currently recommends treatment of ICP over 22 mmHg, which is the threshold that has been associated with increased mortality. Lack of treatment or inappropriate treatment of elevated ICP accounts for the majority of secondary brain damage across a variety of conditions including TBIs, cardiac arrest, and stroke. In TBIs, elevated ICP has been associated with decreased functional status, decreased neuropsychological function (e.g., executive functioning, memory, and information processing speed), and increased mortality across 6-months following a TBI.30 Longitudinal monitoring of ICP is indicated in moderate to severe TBI, as it can substantially improve patient outcomes.

ICP monitoring is well established in the clinical management of moderate and severe TBIs and is increasingly emphasized in cardiac arrest and stroke indications. However, the current standard of care for ICP monitoring is invasive intraparenchymal or intraventricular monitoring. These approaches require drilling a hole through the skull and inserting a catheter directly through brain tissue to monitor ICP. Invasive monitoring is highly accurate, but it carries risks of life-threatening infection (6%) and bleeding (12%). Consequently, invasive monitoring is only indicated in patients with high risk of elevated ICP, which is restricted to moderate to severe TBIs. Of the 3.6 million patients that are affected by neurologic injury, approximately 25% (~900 k patients) are at-risk for elevated ICP, but only 3% of patients that are at-risk receive invasive monitoring.

Through interviews and the surveys of artisans in this field of technology, we determined that clinicians would overwhelmingly prefer ICP monitoring for 94% of patients if there was a safe, accurate, and easy to use approach. For example, non-invasive monitoring of mild, moderate, and severe cases that are not candidates for invasive ICP monitoring would enable intensivists to de-escalate mild and moderate cases sooner, resulting in overall system cost re-ductions associated with ICU length stay and unnecessary CT imaging. Furthermore, non-invasive monitoring would enable earlier identification of patients that require invasive monitoring and immediate intervention due to elevated ICP. Consequently, there is a significant unmet need for innovative non-invasive, continuous monitoring approaches that estimate ICP with high accuracy to reduce complications, increase ICP monitoring in mild and moderate TBI cases, and expand ICP monitoring to additional indications.

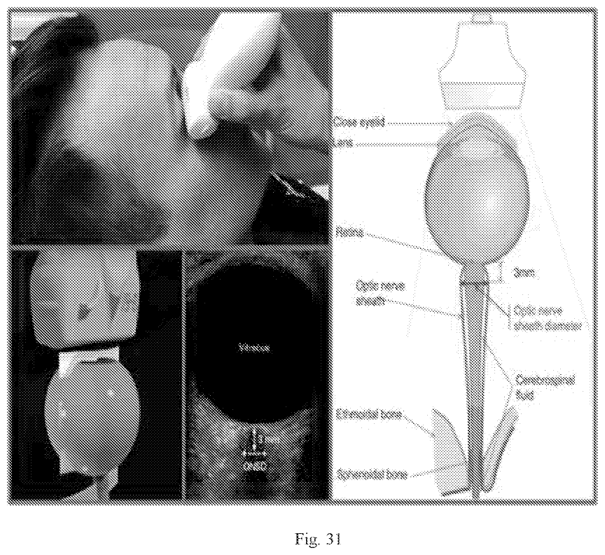

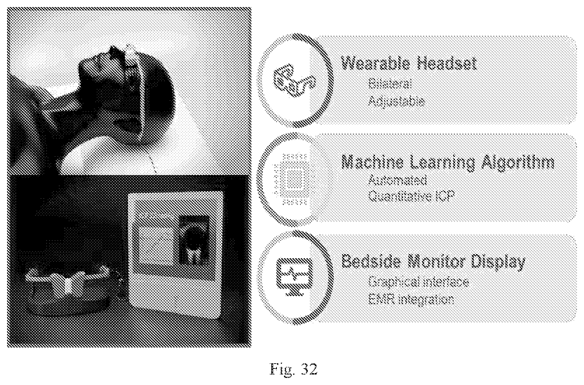

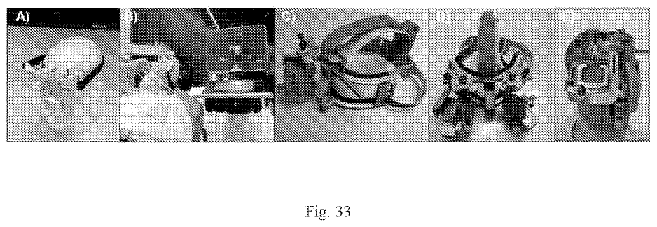

Ocular ultrasound enables non-invasive detection of elevated ICP and holds significant potential for non-invasive, continuous monitoring. Ocular ultrasound is a promising non-invasive approach for assessment of elevated ICP (see FIG. 31). Ocular ultrasound is used to measure the optic nerve sheath diameter (ONSD), where the nerve sheath is contiguous with the subarachnoid space around the brain and the nerve sheath swells as ICP rises. Currently, ONSD is measured manually and a clinical cutoff is used for the ONSD (approximately 5.6 mm), which delivers a binary result of normal or elevated ICP. This approach has become widely recognized for estimating elevated ICP, where recent meta-analyses reported pooled sensitivities and specificities of 90% and 85%, respectively. While ONSD can be used for sensitive detection of elevated ICP using the clinical cutoff, it currently cannot be used to estimate continuous ICP levels, as it shows relatively poor correlation with true ICP levels. Consequently, ONSD cannot currently be used to replace invasive monitoring approaches. However, the technique is relatively simple, requiring little training, and the extensive information that can be gained from analysis of ocular ultrasound images/videos make it a highly promising method for both accurate.

The invention disclosed herein provides accurate non-invasive approaches to estimating ICP which can transform neurocritical care by enabling more patients to receive ICP monitoring, thereby reducing the need for conventional invasive ICP monitoring and associated risks.

### Illustrated Embodiments of Systems and Devices of the Invention

The invention disclosed herein has a number of embodiments. Embodiments of the invention include, for example, hands free systems for estimating intracranial pressure in a subject. As shown in the figures, typically these systems include a headset adapted to rest on the head and/or face of the subject that is operatively coupled to at least one sensing probe. In such systems, the sensing probe can be directly coupled to the headset and adapted to capture a plurality of sensed parameters from a plurality of axes; such that the system can correlate one or more sensed parameters with intracranial pressure so as to estimate intracranial pressure in the subject noninvasively. In some embodiments of the invention, the headset comprises at least one of: a longitudinal member adapted rest on an ear of the subject; a longitudinal member adapted rest on the nose of the subject; a supportive member adapted to rest on the cheek of the subject; a flexible band adapted to secure the headset to the head of the subject; and a deformable material embedded in the headset.

In typical embodiments of the invention, the sensing probe(s) perform at least one process selected from: an ultrasound process; a shear-wave elastography process; a pupillometry process; a dynamometry process; a near infrared spectroscopy process; an acoustic tympanometry process, an optical coherence tomography process; and an electroencephalography process. Commonly in such systems, the sensing probe(s) is coupled to a processor adapted to extract the sensed parameters made by the sensor probe, which change in concordance with changes in intracranial pressure. In certain embodiments of the invention, the processor controls the sensing probe position and orientation in a feedback loop, either through mechanical or electrical means, to optimize data acquisition. In some embodiments of the invention, the sensed data is further analyzed by a separate software algorithm that automatically interprets and quantifies the described measurements. In embodiments of the invention, the system uses these parameters to then estimate intracranial pressure.

In certain systems of the invention, the sensing probe comprises a composition adapted to conduct and receive ultrasonic waves. Typically in such systems, the sensing probe is adapted to capture a plurality of images in multiple planes comprising at least one of: an eyeball transverse diameter; an eyeball anteroposterior diameter; a concavity of an optic disc; a convexity of an optic disc; optic disc elevation; an arclength of a globe; a radius of a globe; an optic nerve diameter, an optic nerve sheath diameter; a deformation, displacement or motion of an optic nerve; corresponding frequency spectra; and/or stiffness of the optic nerve and sheath.

In illustrative system embodiments of the invention, the sensing probe(s) performs an ultrasound process; the system estimates intracranial pressure continuously; and the system comprises a stabilization device that localizes the sensing probe to a region of a patient's anatomy and locks the sensing probe in place at the region of the patient's anatomy. In certain embodiments, the stabilization device of the system comprises one or more adjustable joints adapted to allow the sensing probe to move in a three-dimensional space. In certain embodiments, the stabilization device locks the sensing probe to an eyelid of a patient. In some embodiments, the system is coupled to a processor and uses an autosegmentation algorithm to detect changes in the patient's anatomy.

Embodiments of the invention further include method of estimating intracranial pressure in a subject using a system disclosed herein. For example, some methods of the invention include using the system to estimate intracranial pressure in a subject through an equation or set of equations based on the output from the automated software and demographic parameters of at least one of: age; gender; height; weight, BMI; head circumference; head and brain width; ethnicity; and race. Certain methods of the invention include the steps of modulating or observing physiologic parameters such as at least one of: a body position; a heart rate; a respiratory rate; a systolic blood pressure measurement; a diastolic blood pressure measurement; a mean arterial pressure measurement; a cerebral perfusion pressure measurement; an intracranial pressure measurement; an intraocular pressure measurement; a set of arterial blood gases measurements; and an electrocardiogram tracing. Certain methods of the invention include the steps of observing a comorbid condition(s), such as of at least one of: diabetes; hypertension; cardiovascular disease; cerebrovascular disease; neurologic disorder; ophthalmologic disorder; and smoking status.

In illustrative methods of the invention, the sensing probe(s) performs an ultrasound process. Typically in these embodiments, the method estimates intracranial pressure continuously. Typically in these embodiments, the method uses a system that comprises a stabilization device that localizes the sensing probe to a region of a patient's anatomy and/or locks the sensing probe in place at the region of the patient's anatomy (e.g., on an eyelid of a patient). In certain methods of the invention, a system is designed so that the sensing probe(s) is coupled to a processor adapted to extract the sensed parameters made by the sensor probe, and the system uses an autosegmentation algorithm to detect changes in the patient's anatomy.

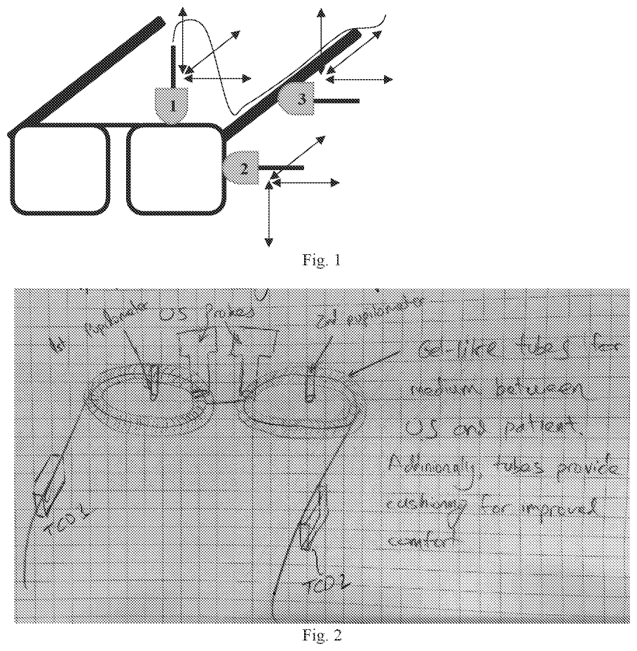

Referring now to illustrative embodiments of the invention in detail, in FIG. 1 there is shown a mountable headset that is worn over the eyes, in similar fashion to standard eyewear, with a plurality of sensing probes at various locations about the frame. Referring now to the invention in more detail, in FIG. 1 there is shown one probe located on the top part of the frame above the eye, a second probe located on the side of the frame beside the eye, and a third probe located on the ear holder. This may also be replicated on the contralateral side. In further detail, still referring to the invention of FIG. 1, the number of probes can be increased or decreased and their locations are adjustable in multiple degrees of freedom about the mountable headset. In further detail, still referring to the invention of FIG. 1, the sensing probes may consist of a variety of sensing modalities including but not limited to ultrasound, Doppler ultrasound, pupillometry, dynamometry, near infrared spectroscopy, acoustic tympanometry, optical coherence tomography, and electroencephalography. Additionally, the sensing modalities (and their probes/adjunct pieces of equipment) are interchangeable and can be removed or added to the headset when needed. In further detail, still referring to the invention of FIG. 1, the sensing modality probe wires may be attached to or routed through the mountable headset (e.g., frame and ear holder).

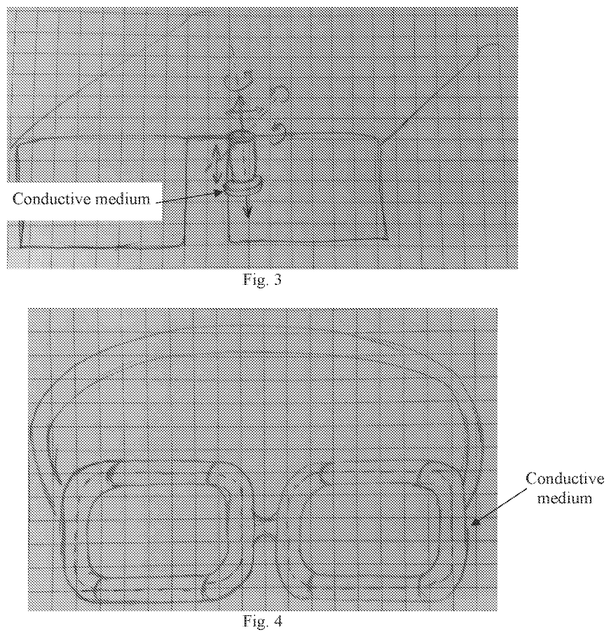

Referring to the invention of FIG. 2, a preferred embodiment of the sensing probes modalities and positioning is shown and is as follows; probe #1 is an ultrasound modality located just below the superior orbital rim and pointing posteromedially towards the optic nerve; probe #2 is a pupillometry modality located laterally to the mountable headset with the ability to extend over the center of the eye for taking a measurement and then retracting away laterally; probe #3 is a TCD modality located laterally to the mountable headset such that it is located over the acoustic window of the temporal bone in the skull.

Referring now to FIG. 3, there is shown an embodiment of the invention in which one such probe can be adjusted in position and orientation relative to the mountable headset and patient with 6 degrees of freedom (three translational axes and three rotational axes) in order to obtain appropriate spatial information. Referring now to the invention in more detail, in FIG. 3 a particular embodiment involves an ultrasound probe sensing modality located just below the superior orbital rim and pointing posteromedially towards the optic nerve. Referring now to the invention in more detail, in FIG. 3 a particular embodiment of the invention uses an ultrasound transducer, which scans a series of two-dimensional images in different planes to sweep out a three-dimensional volume containing anatomic detail of structures of interest within the orbit and optic canal. In further detail, still referring to the invention of FIG. 3 the scanned images can be obtained in axial, sagittal, or coronal planes, or oblique planes in relation to these. In further detail, still referring to the invention of FIG. 3, the images acquired with the probe can be either static (single image) or dynamic (a sequence of images over time). In further detail, still referring to the invention of FIG. 3, the dynamic measurements acquired sequentially over time may include changes of features observed in the aforementioned static measurements, such as deformability or displacement of the optic nerve sheath or posterior globe. In further detail, still referring to the invention of FIG. 3, continuous monitoring with the invention can enable, through dynamic acquisition, the identification of changes in extracted measures in relation to known temporal physiologic patterns, such as with the cardiac or respiratory cycles. In further detail, still referring to the invention of FIG. 3, a medium that allows for conduction of ultrasonic waves is attached to the tip of the probe in order to make continuous contact with body tissue. The conductive medium is flexible and deformable such that it conforms to the external anatomy of a patient. The conductive medium can be a liquid gel or a deformable gelatinous material that can allow for propagation of ultrasound waves without interference. The conductive medium can either be a gel of homogeneous material or a gel/aqueous/liquid/petroleum jelly material encased within a soft polymeric material. In further detail, still referring to the invention of FIG. 3, the conductive medium encased may have the ability to gradually excrete liquid/gel to the probe-tissue interface in order to improve conductivity. In further detail, still referring to the invention of FIG. 3, the conductive medium casing may also be made from hydrogel materials having similar properties to those used for contact lenses, or other radiolucent material.

Referring now to FIG. 4, there is shown a embodiment of the invention in which the conductive medium is affixed to the mountable headset so as to serve both as a cushioning interface between the mountable headset and the patient's body, as well as a medium for ultrasound.

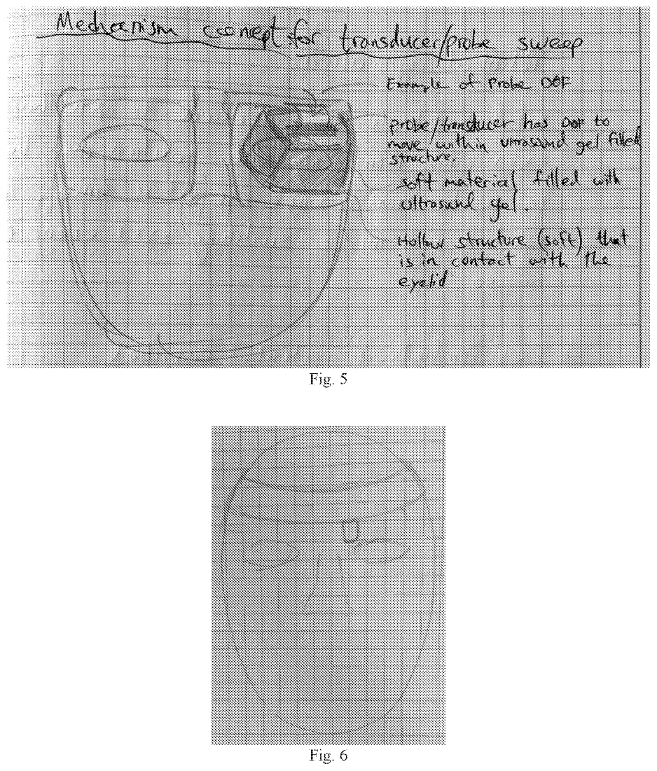

Referring now to FIG. 5, there is shown an embodiment of the invention in which the conductive medium is enclosed in a hollow structure that maintains constant contact with the patient's body. In turn, the ultrasound transducer is contained within the enclosed structure such that the transducer is not in contact with patient and transducer movements have minimal to no effect on contact pressure distribution/location with patient. In further detail, still referring to the invention of FIG. 5, the hollow structure may be a compliant material to promote comfort for patient.

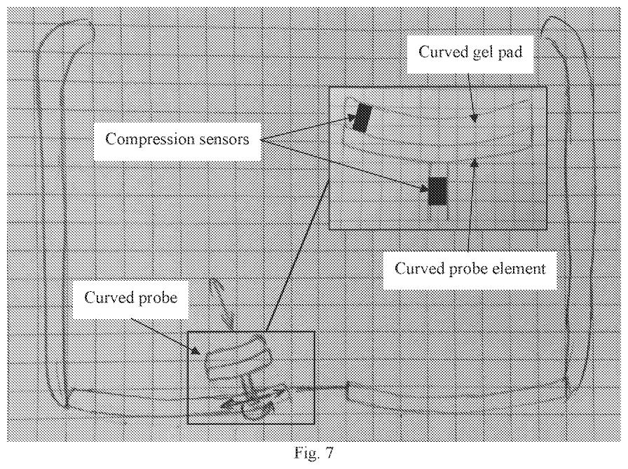

Referring now to FIG. 6, there is shown a embodiment of the invention in which the mountable headset is a strap donned on the head with a probe attached to the strap.

Referring now to FIG. 7, there is shown a top view representation of the mountable headset with a preferred embodiment of an ultrasound probe positioned on the medial part of the device such that it coincides and conforms to the medial canthus. Additionally, the probe would primarily make contact with the sclera of eye but not the cornea, which is more pressure-sensitive. In further detail, still referring to the invention of FIG. 7, there is shown a close-up representation of the curved ultrasound probe and curved gel pad to be in contact with the patient tissue. The probe curvature would approximate that of the globe. In addition, a gel pad with a similar curvature is adhered to the probe and interfaces between the probe and the body. The gel pad helps to prevent any air bubbles forming between the probe and the body and thus obtain a clear image. Furthermore, a variety of sensors, including those measuring strain, pressure, or force, can be embedded between the probe and the frame, the gel pad and the probe, or embedded in the gel pad to detect when appropriate contact strain, pressure, or force are achieved, so as to prevent tissue damage and user discomfort.

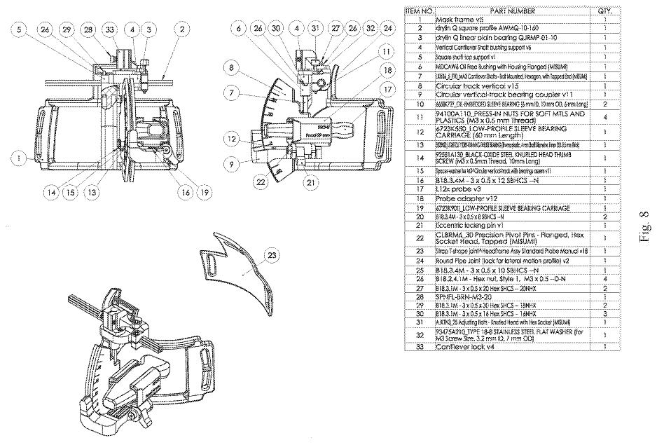

Referring now to FIG. 8, there is shown a specific representation of the mountable headset designed to couple to a standard off-the-shelf ultrasound probe. The headframe allows multiple probe adjustments including translations along the frontal axis, vertical axis, and anterior/posterior direction. The headframe also allows rotation about some vertical axis and about some frontal horizontal axis.

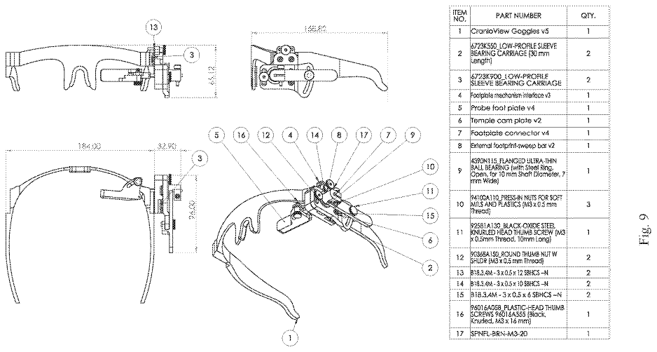

Referring now to FIG. 9, there is shown a specific representation of the mountable headset designed to couple to a custom ultrasound probe. The headframe allows multiple probe adjustments including translations along the frontal axis, vertical axis, and anterior/posterior direction. The headframe also allows rotation about some vertical axis and about some frontal horizontal axis.

Referring now to FIGS. 1-9, a preferred embodiment of the invention involves a mountable headset embedded with nine-DOF inertial measurement unit (IMU) composed of a three-DOF accelerometer measuring linear acceleration, a three-DOF gyroscope measuring angular velocity, and three axis of induction magnetometer. Separate sensors for both the transducer and headframe can be able to collect absolute orientation angles for both the patient head and the transducer.

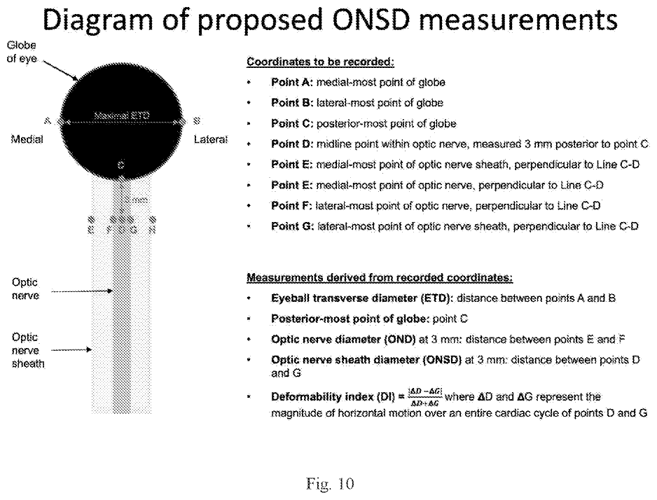

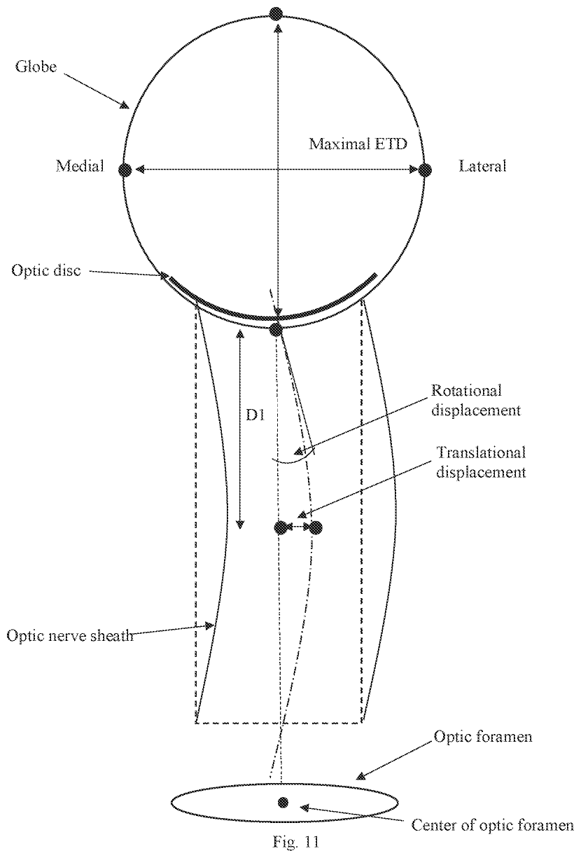

Referring now to FIG. 10, there is shown a representation of the globe, the optic nerve, and the optic nerve sheath. Using an ultrasound probe with the invention, a plurality of anatomical and physiological features in and around the eye can be measured including, but not limited to, the eyeball transverse and anteroposterior diameters, the optic disc elevation, concavity or convexity, the arclength and radius of the posterior globe, the optic nerve diameter and optic nerve sheath diameter along their entire length, the optic nerve and optic nerve sheath perimeter roundness, nerve structure curvature along its length, and the optic nerve and optic nerve sheath cross-section area as well as volume between two cross-sections. Various mathematical ratios or indices can also then be obtained comparing any combinations of these measurements. In further detail, still referring to the invention of FIG. 10, the relative anterior or posterior displacement of the nerve can be measured over time as a change in the globe anteroposterior diameter or as the location of the posterior-most point on the globe (point C).

In further detail, still referring to the invention of FIG. 10, several properties of the optic disc and posterior globe can be measured including, but not limited to, its elevation, relative concavity or convexity, as well as the arc length and radius. In further detail, still referring to the invention of FIG. 10, the optic nerve sheath and adjacent structures stiffness and mechano-elastic properties can be measured via a number of methods including, but not limited to, shear wave elastography, and temporal and/or spectral motion analysis.

Referring now to FIG. 11, there is shown a representation of the globe and the optic nerve being deformed due to changes in ICP. The displacement can be measured relative to the nominal center axis. The nominal center axis can be determined by drawing a straight line between the center of the optic disc and the optic foramen where the nerve emerges from the sphenoid bone. In further detail, still referring to the invention of FIG. 11, the distance where maximum displacement (D1) occurs relative to some fixed anatomy (e.g., the center of the optic disc or center of the globe) can be measured. In further detail, still referring to the invention of FIG. 11, the rotational displacement can be measured as the angle between the nominal center axis and the tangent to the displaced center axis, where the tangent coincides with the point where the two axes cross each other.

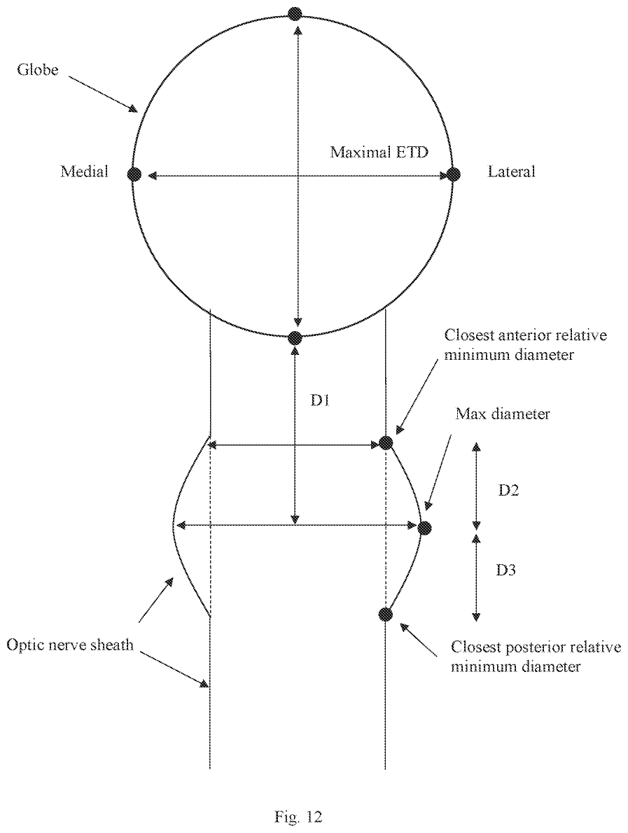

Referring now to FIG. 12, there is shown a different representation of the globe and the optic nerve being altered due to changes in ICP. The displacement of structures can be measured as a maximum sheath diameter and its distance relative to some fixed anatomy (D1). The smallest diameter can also be measured. Additionally, the shortest distances between the relative minimal and maximal diameters in both anterior (D2) and posterior (D3) directions can be measured. In further detail, still referring to the invention of FIG. 10-12, kinematic and motion analysis of the optic nerve sheath and surrounding structures can be carried out in order to associate mechanical and elastomechanical properties to elevated ICP. Methods can include:
a. Spectral analysis of motion in any direction such as anterior/posterior or along the frontal axis, for extraction of first harmonics. Specific harmonics as well as relative amplitudes and frequencies (e.g., between 1^{st} and 2^{nd} harmonics) can be evaluated.
b. Transfer function methods to extract dynamic mechanical properties of the optic nerve and sheath.
c. Perturbation of the optic nerve may be performed for any of the kinematic and motion analysis methods. The perturbation may be external via some energy source including but not limited to low frequency ultrasound or externally induced eye movement. Internal perturbation may also be used, e.g., via voluntary eye movement.

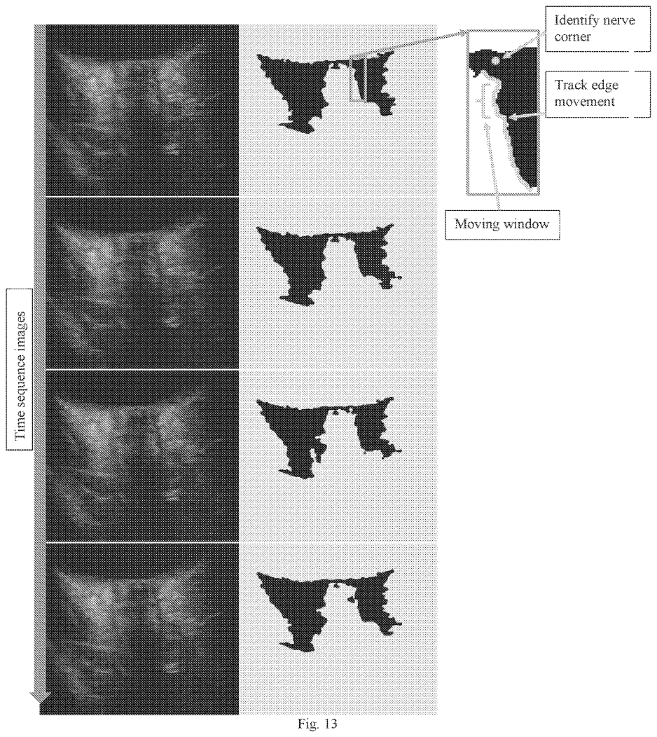

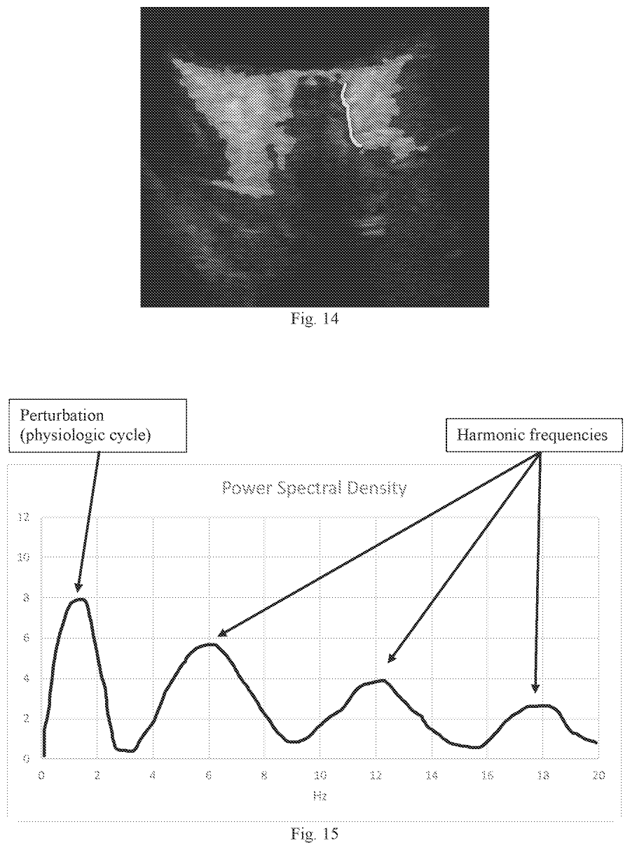

Referring now to FIG. 13-14, for the purpose of spectral analysis, motion tracking of the optic nerve and sheath, can consist of any of the following:
d. Preprocessing of raw images to be fed to a machine learning algorithm for training, which may consist of cropping images to relevant anatomy, adjusting brightness and contrast, filtering (e.g., with median, gaussian, or other filter), converting to a binary image via a threshold function (e.g., Yen, Otsu, etc.), and filling holes and/or cleaning islands (separate bodies in the binary image). Example of time sequenced and preprocessed raw and binary image pairs are shown from top to bottom in FIG. 13.
e. The machine learning algorithm can output accurately segmented images of the optic nerve, optic nerve sheath, and eye, in binary format. Additionally, nerve “corners”, defined as continuation of the optic nerve sheath boundary as it meets the lamina cribrosa, which may need to be estimated if out-of-plane on a given image, are autonomously identified as shown in FIG. 13.
f. Motion tracking can be performed based on the detected edge in the binary images. To analyze motion perpendicular to the optic nerve along the whole imaged nerve, a moving window of pixels along the nerve edge can be used for the calculation as shown in FIG. 13. For consecutive images, respective pixels can be identified either by considering a fixed distance (parallel to the nerve axis) from the nerve corner, an absolute pixel distance from top of the image, or a distance from some reference anatomy.
g. An alternative approach to the above involves overlaying the binary image over the raw image (see FIG. 14) where pixels in the raw image can be selected such that they are along the edge of the binary image. For each selected pixel, motion can be analyzed by incorporating a surrounding area of pixels (region of interest) about the selected pixel. In this method the motion of the selected area of pixels in the image is tracked in consecutive images (e.g., using template matching techniques) and can be considered to represent the motion of the selected pixel.
h. Rather than directly tracking movement, intensity of pixels can be used as a proxy for motion. Similar to the above, a specific pixel or area of pixels can be selected. In this case, the same pixel location or area of pixels are observed over consecutive images in order to track changes in their intensity. Such pixel intensity changes reflect optic nerve and sheath anatomy motion in the transverse, sagittal, or coronal plane, or in a combination of translations and rotations.
i. For an area of pixels, the total intensity can be calculated as a mean intensity or some combination of intensities of the underlying pixels.

Referring now to FIG. 15, periodic perturbations to the optic nerve sheath may be seen arising from local pulsatile flow (e.g., surrounding blood vessels) or respiratory pressure variations related to cerebrospinal fluid flow via Pascal's Law. Consequently, for any of the motion tracking methods above, the frequency content may consist of a fundamental frequency and higher harmonics. The example shows the fundamental frequency at 6 Hz, with the 2^{nd} and 3^{rd} harmonics at 12 Hz and 18 Hz respectively. The perturbing periodic signal is shown as a peak at ~1 Hz (e.g., associated with heart rate). It is hypothesized that motion properties of the optic nerve sheath anatomy are reflective of ICP and can enhance ICP estimation. Thus, it is expected that as ICP increases, there is an increase in the fundamental frequency while the amplitude decreases.

Referring now to FIG. 13-15, image stability is a priority for all the motion analysis methods, including the pixel intensity method. A key objective of the proposed device is to facilitate reliable and stable image acquisition. Image processing methods may be employed in order to identify and consequently reduce unwanted motion (noise) in the image sequence. These may include area-based image registration methods such as Phase correlation, Block matching, and Spatial-temporal gradient, as well as feature-based methods, and probability based methods (e.g., mutual image information).

Referring now to FIG. 16, the pixel intensity method was implemented to ocular ultrasound images collected from healthy subjects. The method involved the following key steps:
j. Measure variance for each pixel during the 15 s sequence of images.
k. Select the 95th percentile pixels with highest variance.
l. Calculate Discrete Fourier transform and power spectral density (PSD) for the selected pixels.
m. Generate plot of PSD which shows power in the signal as a function of frequencies. In the results shown, the peak at 1.604 Hz represents the heart rate. The higher frequency peaks at 7.016 and 10.16 Hz are hypothesized to reflect mechano-elastic properties of the optic nerve sheath tissue. The last peak at 14.43 Hz is assumed to be the second harmonic of the 7.016 Hz peak. As ICP increases the stiffness of the optic nerve sheath increases resulting in change to its natural frequency. Consequently, these higher frequency signals are expected to change with ICP.
n. Superimpose the top-variance pixels on the initial image to highlight anatomically where greatest variability occurred. It is expected that the pixels with the greatest variability can reside within or about the optic nerve sheath as is the case in the superimposed image.
   In further detail, still referring to the invention of FIG. 16, the pixel intensity method can be thought of as multiple, simultaneous A Mode ultrasound or an area at a certain depth, where each pixel is a separate A Mode. Tracking each pixel over time results in amplitude variations due to tissue changes at that depth.

Referring now to FIG. 17, translation motion (in x and y directions) of the area of pixels selected in FIG. 16 was also estimated using intensity-based registration. This resulted in x and y motion estimates. Consequently, PSD was calculated for both the x and y direction motions. The figure shows that it is possible to detect the low and high frequency peaks in the y axis PSD but not in the x axis. It should be noted that this technique was generally less reliable than the pixel intensity method in identifying the frequency peaks seen in the FIG. 16.

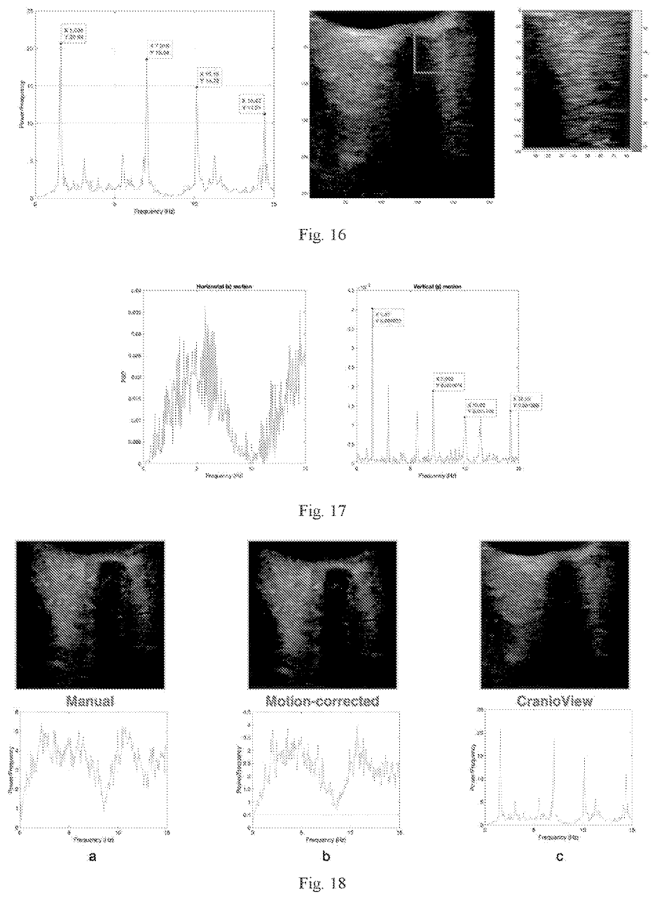

Referring now to FIG. 18, the pixel intensity method was implemented to manual ocular ultrasound recording from healthy subjects and compared to recording with the device in this invention. Image a was recorded manually, image b is the same manual recording but with image registration to reduce unwanted motions, while image c was recorded with the device in this invention. All the images were processed with the same pixel intensity method described above. It can be seen that there is significantly more noise in the manual recorded images and that in this case the image registration failed to distinguish the desired high frequency signals seen in FIG. 16.

Referring now to FIG. 13-18, a key element of the present invention is the combination of the wearable device which provides image stabilization, and the image processing methods. The ability to identify and resolve dynamic signals (e.g., low, and high frequency motions) in the imaging is facilitated by first, the wearable stabilization device which limits motion artifacts and noise in the sequence of images, and second, the image processing methods (e.g., pixel intensity) applied to the stabilized sequence of images.

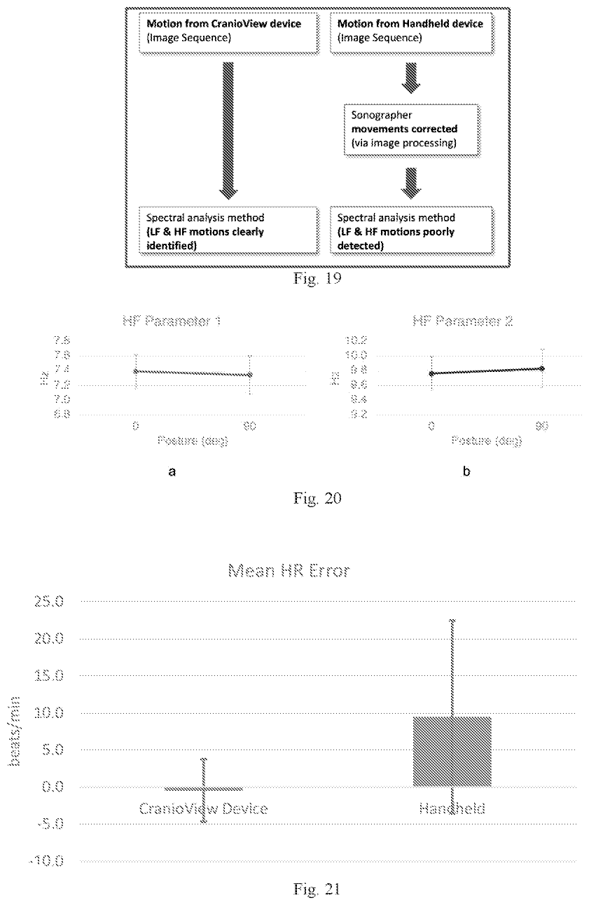

Referring now to FIG. 19, a diagram depicting the process for the spectral analysis implemented for an ultrasound recorded with the device in this invention or manually. For FIG. 18 c, the motion sequence is directly processed with spectral analysis whereas for FIG. 18 a, the motion sequence is subject to motion registration and correction before spectral analysis processing. In the former, both low frequency (LF) and high frequency (HF) are more readily identifiable.

Referring now to FIG. 20, subject data was recorded in different postures including supine (0 degrees) and upright (90 degrees). HF parameters 1 (referring to ~7 Hz signal in FIG. 16) and HF 2 (referring to ~10 Hz signal in FIG. 16) are shown for each posture. Opposing trends are observed for HF1 and HF2 between supine and upright postures. This data is from healthy subjects who are not suffering from elevated ICP. However, it is expected that this or another relationship can become more pronounced by processing patient data.

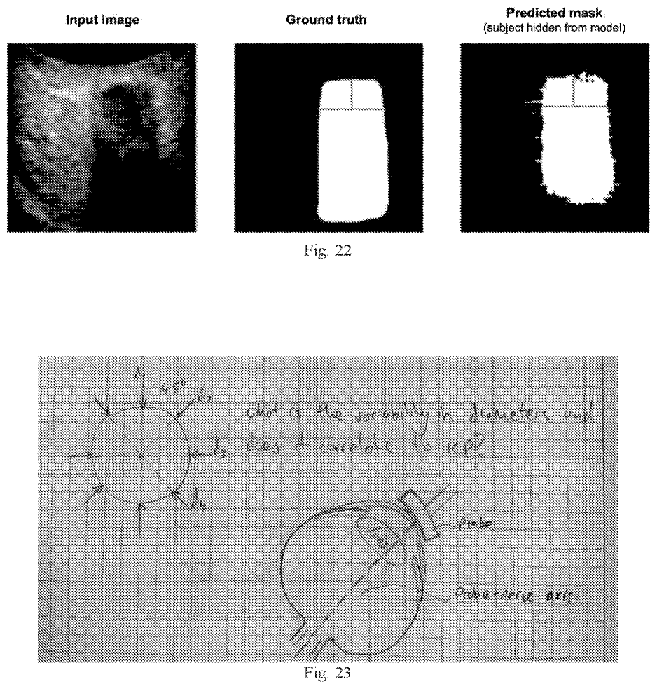

Referring now to FIG. 21, subjects heart rates were also recorded physically (pulse oximeter). Errors between the heart rate extracted from the ultrasound recording (using the pixel intensity approach) as shown in FIG. 16 and the physical recording were compared for the device in the current invention and a standard manual ultrasound recording. FIG. 21 shows errors were significantly smaller for the device in the current invention.

Referring now to FIG. 22, an autosegmentation algorithm is required in order to automate measurements of the optic nerve sheath (ONS). To perform the auto segmentation, we have utilized an existing modified U-Net model provided by Tensorflow. U-Net is a novel deep learning tool based upon a convolutional neural network that was developed for use in biomedical image segmentation. We have created a data pipeline to interface with this existing model that accepts the ocular ultrasound dataset of images and labeled binary masks of the ONS as inputs. The Machine Learning model was trained on data from healthy subjects. The figure shows the input image, its manually labelled ground truth, and the model output for an unseen subject. The predicted output mask shows the model successfully identifying the optic nerve sheath. The ONSD error between the model output and the ground truth was about ~3.3%. In further detail, still referring to the invention of FIG. 22, future versions of the machine learning model can not only rely on raw and labeled images but also on surrogate features including but not limited to the ONSD, Globe transverse and anteroposterior diameters, optic disc elevation, curvature or radius, optic nerve sheath motion velocity, frequency, and stiffness in order to train the model and predict ICP with high accuracy.

Referring to FIG. 23, there is shown a general anatomy of the eye and optic nerve with an axis defined between the optic nerve and an ultrasound transducer (probe-nerve axis). Imaging of the optic nerve and sheath may be obtained in different orientations such as transverse, sagittal, and additional angles between. In further detail, still referring to the invention of FIG. 23, in one embodiment the optic nerve and sheath diameters may be captured in multiple angles (e.g., 30-degree increments) about the probe-nerve axis. The approach may be beneficial in providing an area approximation of the optic nerve and sheath at a particular cross-section and volume over a certain section of the optic nerve and sheath, which may be more sensitive to changes in ICP compared to, e.g., a diameter in only the sagittal or the axial plane. Additionally, the roundness of the cross-section may also contribute to ICP correlation. For two diameters, the roundness may be evaluated as Roundness=(Dmax−Dmin)/2, where Dmax is the maximum diameter value and Dmin is the minimum diameter value. A roundness=0 would imply perfect roundness.

Referring now to FIG. 10-23, the following measures are considered that are hypothesized to contribute to significant correlation with ICP:
a. Optic nerve sheath diameter changes in relation to ICP changes;
b. Optic nerve diameter changes in relation to ICP changes;
c. Optic nerve sheath cross-sectional area changes in relation to ICP changes;
d. Optic nerve cross-sectional area changes in relation to ICP changes;
e. Optic nerve sheath volume changes in relation to ICP changes;
f. Optic nerve volume changes in relation to ICP changes;
g. Globe transverse and anteroposterior diameters change in relation to ICP changes;
h. Globe and optic disc elevation, curvature and radius changes in relation to ICP changes;
i. Diameters along the entire length of the optic nerve and optic nerve sheath as they change in relation to ICP changes;
j. Nerve structure maximal displacement changes in relation to ICP changes;
k. Location (D1) of nerve structure maximal displacement changes in relation to ICP changes;
l. Anterior (D2) and posterior (D3) distances between maximal and closest relative minimal diameters in relation to ICP changes.
m. Frequency and amplitude of first few harmonic frequencies associated with the optic nerve and sheath stiffness and movements
n. Velocity associated with the optic nerve and sheath movements when exposed to shear-wave elastography

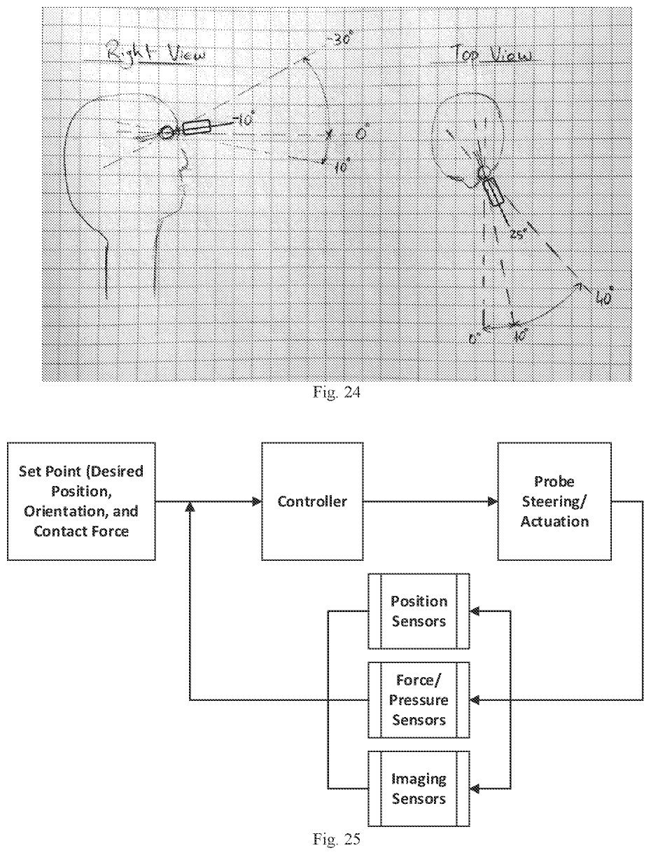

Referring now to FIG. 24, a preferred embodiment of the invention is shown with an ultrasound probe that is angled similarly to the resting anatomic position of the optic nerve. Experimentally, when placing an ultrasound just below the superior orbital rim, this is typically found at about 10 degrees below neutral in the vertical axis (with a full ultrasound sweep ranging from +10 degrees to −30 degrees in relation to the vertical axis) and about 25 degrees directed medially in the horizontal axis (with a full ultrasound sweep ranging from +10 degrees to +40 degrees in relation to the horizontal axis).

Referring now to FIG. 25, there is shown a representation of a closed-loop system. Given a control goal defined by a desired position and orientation, contact force or pressure, and parameters defined by the various sensed modalities, a controller can command the device actuation and steer the probe to achieve the desired control goal.

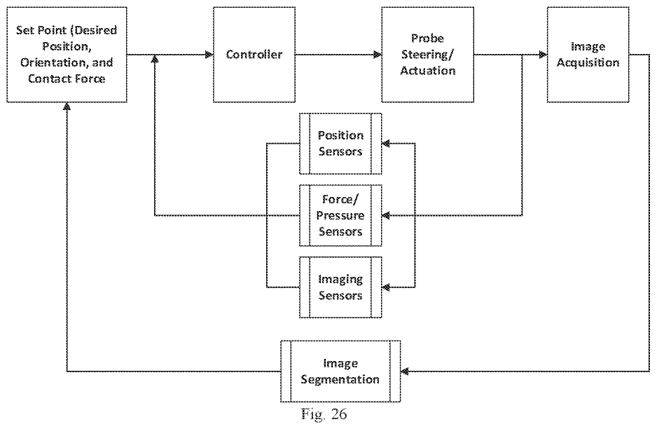

Referring now to FIG. 26, there is shown a specific expansion of the closed-loop system from FIG. 18. Following the actuation, the probe takes an image which in turn is segmented by a processing unit. If quality of the segmented image is insufficient, the control goal is updated and fed back into the closed loop in FIG. 25 to result in a change in set point. In further detail, still referring to the invention of FIG. 25 and FIG. 26, the probe actuation may involve mechanical steering, where the probe is physically moved, or electronic steering, where the probe signal direction can be electronically adapted. In further detail, still referring to the invention of FIG. 25 and FIG. 26, the controller may be a position, force, or hybrid position-force controller.

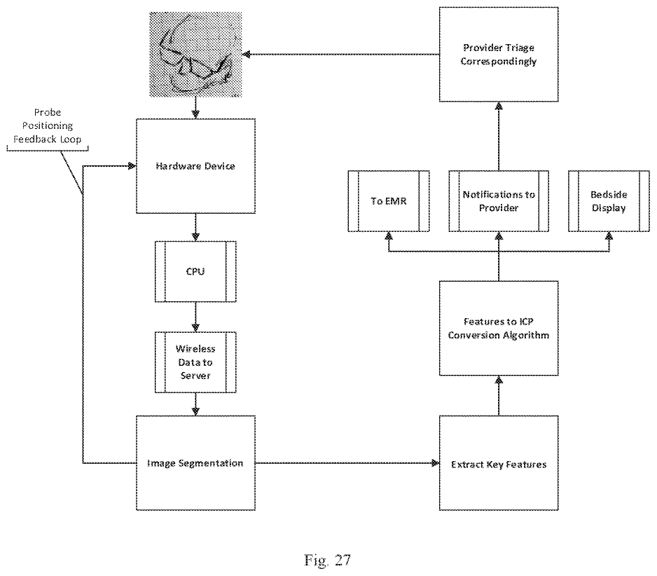

Referring now to FIG. 27, there is shown a representation of a closed-loop system implementation in a clinical setting involving a patient, the current invention, and a healthcare provider. The diagram describes the following process: the mountable headset is donned on the patient; the probes on the mountable headset acquire images of the desired anatomy; the raw data is processed and converted to digital format; the digital data is transferred to a server or a remote computer, either through a wired or wireless connection; the acquired images are segmented in order to extract important features as described in FIG. 10-26; based on the important features and additional physiological information, an algorithm calculates a value for ICP; the generated data is stored and displayed in the patient's electronic medical record, displayed on a bedside monitor, and/or sent as a notification to the provider; the provider can then make appropriate triaging decisions. In further detail, still referring to the invention of FIG. 27, in order to most accurately estimate ICP, the algorithm can also integrate a variety of physiologic parameters including but not limited to blood pressure, heart rate, respiratory rate, arterial waveform, arterial blood gas, blood oxygen saturation, brain tissue oxygen saturation, brain metabolite microdialysis, jugular venous bulb oxygen saturation, intraocular pressure, pupillometry, transcranial doppler, and electroencephalography; as well as patient demographics including but not limited to height, weight, BMI, head circumference, head and brain width, gender, race, age, and health comorbidities.

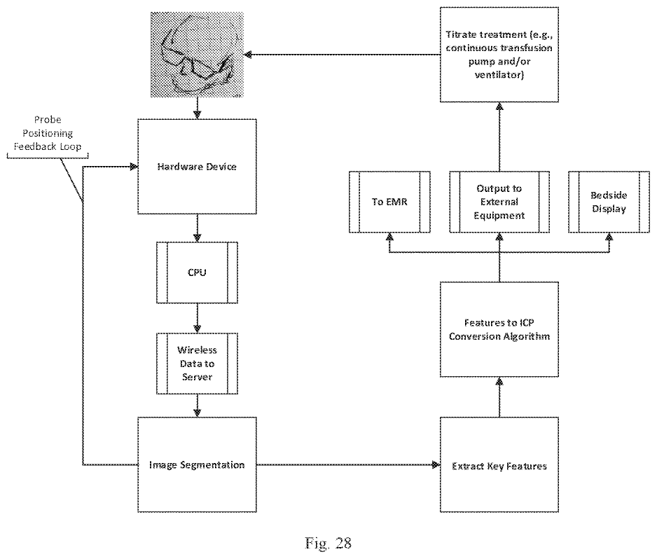

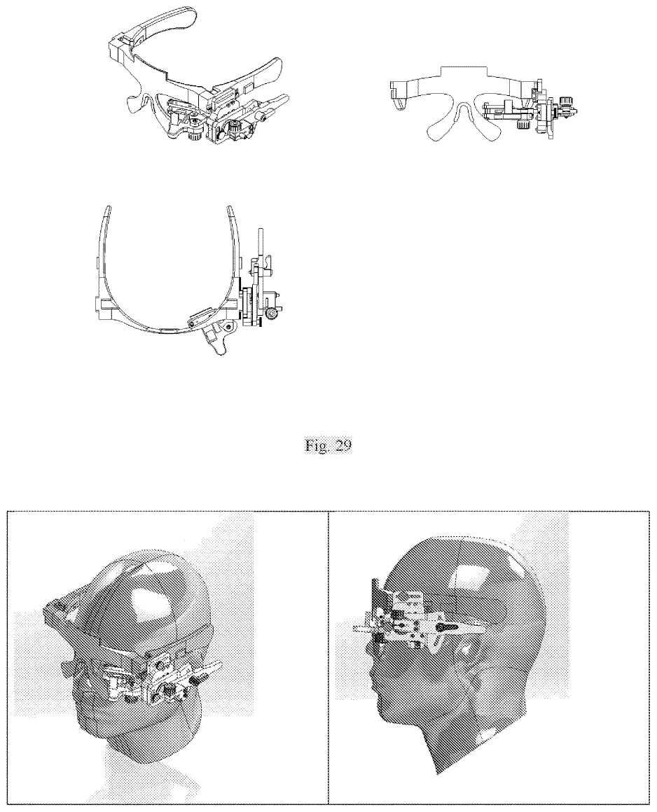

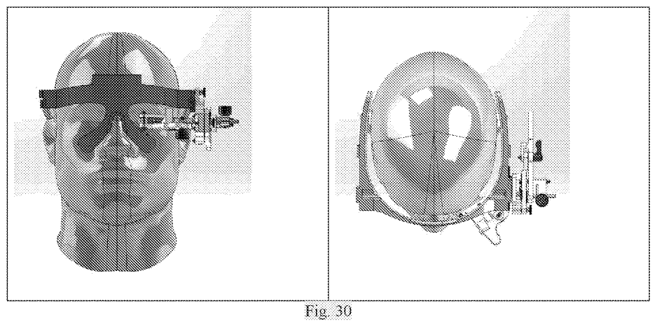

Referring now to FIG. 28, the closed-loop system may interface with existing medical equipment such as a continuous infusion pump or a ventilator, so as to automatically titrate medical treatments. In this scenario, the loop involves a patient, the current invention, and external medical equipment.

The advantages of the present invention include, without limitation, continuous and autonomous image acquisition (with ultrasound and other modalities) and over long periods of time for a given individual. This can allow for personalized measurement trends on a per-patient basis.

The invention can either autonomously or by operator command be able to select and utilize a subgroup of measuring modalities that result in optimal ICP estimation. The software algorithm component of the invention can have the ability to process data from each of the independent measuring modalities, and combine those inputs to derive an accurate estimate of intracranial pressure, which can be the ultimate output that clinicians can use to make clinical decisions.

The software can then use the optimal images acquired and be able to, either through machine learning methods or through standard image processing methods, segment anatomic structures of interest to derive one-, two-, and three-dimensional relationships between structures, and of structures over time. In this way, the data acquired, e.g., by an ultrasound probe, and extracted through the software processing can be both static and dynamic, and may be based on specific mathematical ratios and/or indices.

The software invention can have the capability, after anatomic segmentation, to correct the ultrasound probe positioning and parameters (e.g., gain, frequency, etc.), through feedback loops, to identify an optimal ultrasound probe positioning and parameters that result in acquiring optimal images.

Incorporating this sensor-based data, including data derived after software processing, with other patient parameters and demographic data, a method and algorithm can be able to yield accurate estimation of ICP with pertinent confidence intervals.

This intracranial pressure estimation may be plotted in graphical format and displayed such that an operator can interact with the data.

### Illustrative Aspects and Embodiments of the Invention

In broad embodiments, the present invention is a wearable, non-invasive, autonomous, and continuous measuring system comprised of hardware, software, and a custom method for estimating ICP. In brief, the invention provides a combination of hardware and software which combines multiple sensing modalities and patient parameters and demographic data to estimate ICP noninvasively and continuously. The hardware component consists of a wearable apparatus with components resting about the patient's head and face much like one would wear a pair of glasses. The principal imaging component can typically be an ultrasound probe resting below the superior orbital rim of the eye, which can take a series of images in multiple planes to capture both the eye and the optic nerve sheath. The raw image is fed into an image-processing software program that may function either through machine learning or through traditional image processing methods to automatically segment specific measurements (some well-studied, others more experimental) which change in concordance with changes in ICP.

The invention disclosed herein has a number of embodiments. In certain embodiments of the invention, the headset comprises at least one of: a longitudinal member adapted to rest on an ear of the subject; a longitudinal member adapted to rest on the nose of the subject; a supportive member adapted to rest on the cheek of the subject; a flexible band adapted to secure the headset to the head of the subject; and/or a deformable material embedded in the headset.

In certain embodiments of the invention, the system is coupled to a sensing probe and a processor adapted to perform at least one process selected from: an ultrasound process; a pupillometry process; a dynamometry process; a near infrared spectroscopy process; an acoustic tympanometry process; an optical coherence tomography process; and an electroencephalography process. In certain embodiments of the invention, the sensing probe or probes may be coupled to an actuator adapted to position the sensing probe.

In certain embodiments of the invention, the sensing probe comprises a composition adapted to conduct and receive ultrasonic waves; is adapted to capture a plurality of images in varying planes; is adapted to capture images of the eye, the optic nerve sheath, and the surrounding structures. In such systems, the sensing probe is coupled to the headset in an orientation such that the sensing probe can sense at least one of: an eyeball transverse diameter; an eyeball anteroposterior diameter; optic disc elevation, a concavity of an optic disc; a convexity of an optic disc; an arclength of a globe; a radius of a globe; an optic nerve diameter; an optic nerve sheath diameter; and deformation or displacement of an optic nerve. In certain embodiments of the invention, the system is coupled to a processor adapted to extract sensed parameters made by the sensor probe which change in concordance with changes in ICP.

In certain embodiments of the invention, the sensed data and software-derived data may be supplemented by an individual's demographic parameters, of at least one of: age; gender; height; weight; ethnicity; and race; or physiologic parameters of at least one of: a body position; a heart rate; a respiratory rate; a systolic blood pressure measurement; a diastolic blood pressure measurement; a mean arterial pressure measurement; a cerebral perfusion pressure measurement; an intracranial pressure measurement; an intraocular pressure measurement; a set of blood gas measurements; and an electrocardiogram tracing; or comorbid conditions of at least one of: diabetes; hypertension; cardiovascular disease; cerebrovascular disease; neurologic disorder; ophthalmologic disorder; and smoking status.

In certain embodiments of the invention, different mathematical methods may be used to incorporate the sensed and processed data to estimate an individual's ICP. The ICP can be displayed as the output to a bedside monitor for interpretation by the provider, and may also push relevant data points into the electronic medical record or provider's cellular phone for customizable notifications.

As disclosed above, embodiments of the invention further include methods of making and using the systems disclosed herein.

### EXAMPLE 1: ILLUSTRATIVE DEVICES AND SYSTEMS FOR MEASURING ELEVATED INTRACRANIAL PRESSURE

Embodiments of the invention include ultrasound hardware and software designed to improve neurocritical care outcomes. Embodiments of the invention can include a wearable, non-invasive ICP measurement system that employs a stabilization device to improve ultrasound measurement and to enable continuous monitoring. Embodiments of the invention also include machine learning (ML) algorithms that support automatic extraction of ONSD, surrounding anatomical structures, and dynamic features to create a multivariate model for improving non-invasive ICP estimation. In our studies, we have demonstrated equivalence between embodiments of the invention and a machine learning (ML) measurement approach through a study of 12 healthy subjects and ~1,000 images. In embodiments of the invention, an autosegmentation approach was able to automatically identify and measure the ONSD with 97% accuracy of expert measurement, a finding which demonstrates the feasibility of continuous, real-time optic nerve sheath segmentation. We also demonstrated that the stabilization device significantly reduces motion artifacts acquired during ultrasound measurement, which enables accurate identification of dynamic physiologic signals, such as brain tissue motions, toward improving ICP estimation accuracy. In this way, embodiments of the invention leverage high-density time-based monitoring, extracting novel dynamic ICP surrogate features in the ONS, and leveraging advanced ML modeling techniques such as decision trees to optimize the model regression error using combinations of a large number of features (i.e., ONSD+additional dynamic features) to improve estimation of absolute ICP.

We can validate various ultrasound stabilization device embodiments of the invention in clinical studies. We can demonstrate that the stabilization device delivers comparable or better image quality than freehand ultrasound in adult patients admitted to the neurocritical care unit (NCCU) that can be conducted through a qualified contract research organization (CRO). Participants (n=78) that are at high-risk for elevated ICP can be included, where we can focus on TBIs and acute stroke as initial subpopulations at-risk for elevated ICP. Ultrasound images can be collected with both the stabilization device and freehand. Invasive ICP measurements can also be collected in conjunction with ultrasound imaging. We can demonstrate comparable ONSD from embodiments of the invention and freehand ultrasound images as interpreted by an expert and will compare relative time that the image stays focused across the two methods. Quantitative endpoints: ONSD pairwise difference <0.2 mm; total time image can be focused with embodiments of the invention >freehand.

We can optimize and validate ML algorithm for automatic segmentation and interpretation of ultrasound images. Using the image data collected as discussed above, we can validate the ONSD autosegmentation algorithm and can extract and integrate dynamic signals from the time series data such as the deformability index, motion frequency of the optic nerve sheath anatomy, and demographic data to improve ICP estimation. We can optimize and test both deep learning and regression models that integrate these parameters. The Dice coefficient can be used to guide autosegmentation model optimization. We can demonstrate that dynamic parameters with ONSD perform better than ONSD alone. An 80/20 training and test split can be used in addition to k-fold cross-validation to validate the ICP estimation model. Quantitative endpoints: Dice coefficient ≥0.85; Bland Altman analysis; correlation, AUROC, sensitivity, and specificity associated with dynamic parameters≥ correlation, AUROC, sensitivity, and specificity associated with ONSD, respectively.

Embodiments of the invention are expected to improve monitoring and clinical management of ICP toward earlier identification and earlier treatment of elevated ICP to improve morbidity and mortality outcomes. Embodiments of the invention will also enable more patients to undergo ICP monitoring and may eliminate the need for some patients to undergo invasive ICP monitoring.

### ICP Estimation and Continuous Measurement.

Embodiments of the invention comprise devices and systems for non-invasive, real-time ICP estimation and the scientific premise for a multivariate machine learning (ML) approach for ICP estimation using Nondynamic parameters. An illustrative system which includes an innovative wearable for continuous ocular ultrasound-based ICP monitoring is shown in FIG. 32 and that ML algorithms to support automated and improved ICP estimation through integration of additional dynamic parameters that can be resolved with ocular ultrasound. Embodiments of the invention can include an autosegmentation algorithm to accurately identify and measure the ONS from ultrasound images. A central goal is to leverage high-density time-based monitoring, extracting novel dynamic ICP surrogate features, and leveraging advanced ML modeling techniques such as decision trees (e.g., random decision forest and Extreme Gradient Boost) to optimize the regression error using combinations of a large number of features. The mechanism of action of ONSD as a surrogate for ICP due to its anatomical continuity with the dura of the brain and the subarachnoid CSF is relatively well understood. Nevertheless, reports over the last two decades and particularly in recent years have not come to a consensus on the clinical utility of ONSD for continuous ICP estimation. This likely stems from both inconsistent methodologies as well as effects such as baseline variability and hysteresis in the ONS. These limitations possibly resulted in a tendency to focus on ONSD's utility as a binary assessment rather than a continuous estimate. Embodiments of the invention can include address these limitations and take an approach that goes well beyond static ONSD measurements. An illustrative differentiating element to one approach can be the incorporation of transient phenomena in the ONS and surrounding structures. Moreover, by utilizing a wearable device and automating clinical processes, it is highly likely that our system will be able to mitigate methodological deficiencies related to skills and observer variability. Accordingly, there are several sources of evidence that support this combinatorial ML-based embodiments of the invention for improved ICP estimation:

(1) Measuring ONS Stiffness-A Normalized Parameter. The ONSD baseline can vary without relation to ICP. Yet, from a first principles perspective, the stiffness of the ONS increases with pressure.^{1,36} The tissue resonance response associated with stiffness is expected to be far less sensitive to baseline dimensional variations among patients.^{11,13,37} Furthermore, observing individual components in the frequency domain can solve time distortion dynamics that exist in the time domain,¹¹ such as the ONS's potential lagged diameter response. Thus, dynamic signals associated with the ONS stiffness may be considered to be decoupled from patients' specific ONS physiological dimensionality and, overall, more directly reflect pressure.
(2) High Density Temporal Measurements and Feature Extraction for ML Application. There is a lack of research involving time-based signals and associated transient features with ultrasonography through the eye. Hirzallah et al. found that most studies do not report longitudinal testing with multiple measurements over time.³⁸ We identified a limited number of articles performing serial ONSD measurements, however, in the majority of these studies the data was collected over a span of days and hours between readings.³⁹⁻⁴⁹ This is in stark contrast to the density of time-based measurements of embodiments of the invention, which would allow sub second dynamic signal resolution. The most densely collected serial ONSD measurements were reported in a Klinzing et al. article where they reliably detected the dynamic changes in ONSD during short-term hyperventilation in patients with severe TBI and used four serial ONSD measurements to monitor the induced changes within a short time span (~1 hour).⁵⁰ Chopra et al. collected three ONSD measurements before and during a CSF diversion surgery and additional 7 measurements 12 hours postoperatively.⁵¹ Chen et al. recorded two measurements, both 5 minutes before and after lumbar puncture showing an immediate drop in ONSD.⁵²

Recent reports of several non-invasive ICP estimation approaches have specifically looked at data in the form of time series sampled with high temporal frequency from which the researchers were able to extract time-based features for ICP estimation. These approaches include optical modalities such as photoplethysmography with ophthalmodynamometry, diffuse correlation, and near-infrared spectroscopy to measure microvascular cerebral blood flow. Limits of agreement reported on a validation set ranged between 3.8-7 mmHg with 95% confidence limits compared to standard invasive ICP monitoring. Although, the reported results in these articles are promising, signals from optical modalities that observe vascular pulsations in the brain's vascular tissue can be limited by the depth of the vasculature in adults or additional mechanisms that influence the vasomotor tone.

We believe that physiological dynamics can similarly be detected from high density temporal recordings with our ultrasound device. Additionally, with increasing volume of data collected with high sample rate recordings and with the potential to extract new features representing physiological dynamics, ML methods can be highly effective in identifying regression models with invasive ICP data.⁵³⁻⁵⁵ Yet, the work by Miyagawa et al. who developed a supervised ML classifier to predict suspected increased ICP based on CT measurements is the only article identified employing ML methods with ONSD data to predict ICP levels.⁵⁶ This demonstrates a clear scientific gap and a need to conduct further studies based on state-of-the-art ML approaches.

(3) Adjunct Parameters. Numerous studies have explored adjunct parameters either in isolation or in combination with ONSD to improve correlation with ICP. These parameters can be readily combined with our method and include eyeball transverse diameter,^{7-9,57-60} optic disc elevation,^{35,61,62} shear wave elastography,⁶³⁻⁶⁷ and the de-formability index (measure of lateral motion of the optic nerve which is correlated with stiffness and ICP).^{1,36} The use of multivariate models for ICP elevation has significant potential to improve estimation accuracy.³⁵
(4) Embodiments of the invention are capable of capturing transient and dynamic signals. This system has shown capability to accurately capture fine dynamic motions in the ultrasound images. We have demonstrated accurate capture of the heart rate (HR) and high frequency motions that have not been previously reported with ocular ultrasound. However, similar high frequency signals in the 0-30 Hz range have been described in relation to invasive ICP monitoring signals and acoustic/ultrasonic signals.^{11-13,37,77,78} In particular, Goto et al. suggested the natural resonance frequency (NRF) of the brain only depends on the ICP and resided around 20 Hz.¹³ Kim et al. conducted spectral analyses of continuous ICP waveforms during CSF infusion test within the bandwidth of 0.2-15 Hz.¹² Michaeli et al. performed ultrasonography at the third ventricle-brain interface and found that ICP is dependent on the value of the dominant secondary (mechanical) resonance level of brain tissue which varies in the range of 4-30 Hz.³⁷ Bray et al. analyzed the frequency “fingerprint” of the brain and postulated the high frequency components (4-15 Hz) reflect compliance in the brain.⁷⁸ Embodiments of the invention have identified dynamic signals around the ONS in the 5-15 Hz range. It is necessary to collect additional time series of ONS motions in patients to further analyze them in relation to ICP and extract features that can improve ICP estimation.

Several alternative non-invasive ICP estimation approaches are in development, where devices are outlined in Table 1. Currently, the most promising approach for non-invasive ICP estimation is Nisonic's transorbital ultrasound, which has demonstrated good clinical performance for elevated ICP, but cannot be used for continuous ICP estimation. The most studied alternative approach is the use of transcranial doppler ultrasound. The Lucid MI transcranial doppler ultrasound and NeuralBot by NovaSignal can be used to analyze changes in flow patterns of arteries in the brain. It is not currently cleared by the FDA for use in ICP monitoring but is used off-label. Across 19 studies, transcranial doppler has shown varying correlation with ICP (Interquartile range of 0.36-0.8 and median of 0.53),⁷⁰ which is lower than that of ocular ultrasound. There are also an estimated 15% of patients that have a thick temporal bone that are not candidates for transcranial doppler⁷⁹ and, like typical ocular ultrasound, it requires a skilled operator and time to find the appropriate acoustic window.⁸⁰ Near-infrared sensors are also in development with the intracranial pressure assessment screening system (IPASS) by Vivonics, Inc, that measure blood oxygenation through near-infrared sensors on the head. This approach enables continuous, non-invasive monitoring, but there are few studies and accuracy has been criticized. One study demonstrated a correlation of r=0.55, which is below that of ocular ultrasound. Finally, dynamometry with ophthalmic imaging is used to measure blood pressure in the central retinal vein. Third Eye Diagnostics is developing the Cerepress device. Correlation with ICP has been good, ranging from 0.662 to 0.98.72-74 However, this approach is also not continuous and involved direct contact force with the cornea for measurement, which is likely to be uncomfortable or may result in eye injury. Ocular ultrasound has demonstrated good correlation with ICP, and it is more established in the field.

ML and ONSD autosegmentation competition. Modern advances in ML have significant potential to enable autosegmentation of the optic nerve and measurement of ONSD for automatic and improved interpretation of ocular ultrasonography images. Furthermore, ML methods can potentially improve ICP estimation through inclusion of additional parameters. Autosegmentation of ONSD and ML approaches for improved ICP estimation are emerging but are yet to be commercialized. Nisonic is developing an ML approach for autosegmentation of the optic nerve. This can improve inter-observer variability by standardizing the ONSD interpretation. There are also several examples of approaches that use ML methods such as deep learning for autosegmentation of the ONSD from ultrasound images^{81,82} and computed tomography (CT) screening images.⁸³ However, we are not aware of other entities advancing these approaches toward commercialization.

We have completed development of illustrative embodiments of the wearable and autosegmentation algorithms. We have successfully demonstrated feasibility for image stabilization using the wearable and highly accurate autosegmentation and measurement of the ONSD in a cohort of healthy participants. This includes 97% agreement between ONSD derived from the autosegmentation algorithm and ONSD measured by a clinical expert. This strongly supports optimization and validation in patients that are at-risk and present with elevated ICP. We have also demonstrated accurate measurement of heart rate and dynamic signals in ultrasound videos with our image processing facilitated by substantial reductions in noise through the use of the stabilization devices disclosed herein. Furthermore, we have identified high frequency signals that are likely associated with brain tissue dynamics that have not been previously described with ocular ultrasound in the literature. There is significant potential for integration of the ONSD and dynamic parameters into a model for improved ICP estimation, which is facilitated by the improved image quality associated with the stabilization device. The goal of this will be to 1) demonstrate improved image quality and collect a critical ultrasound image dataset in the target TBI population. 2) Complete technical validation of the autosegmentation algorithm in this patient population toward a continuous, non-invasive monitoring approach. 3) Optimize and validate a computational model that integrates ONSD and dynamic parameters for improved ICP estimation.

Embodiments of the invention can transform ICP monitoring through development of novel hardware and software solutions, which enables non-invasive continuous monitoring of ICP with clinical performance at or beyond what is currently available. Embodiments of the invention can significantly increase the number of patients that could receive ICP monitoring toward identifying elevated ICP or clinical events that require immediate intervention earlier. This is expected to significantly improve morbidity and mortality outcomes for neurocritical care unit (NCCU) patients.

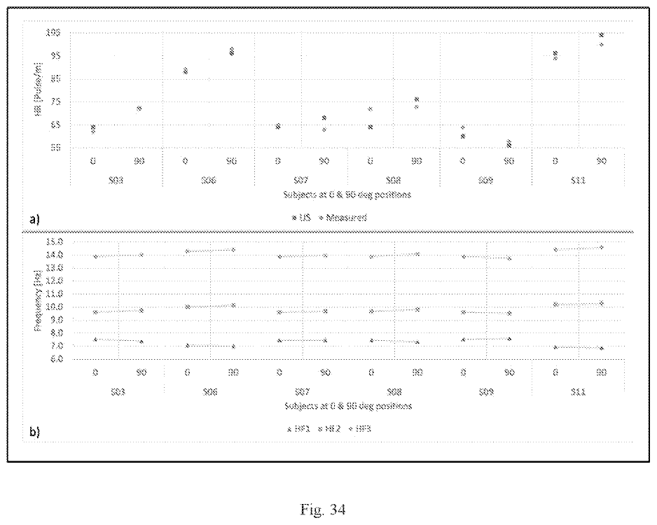

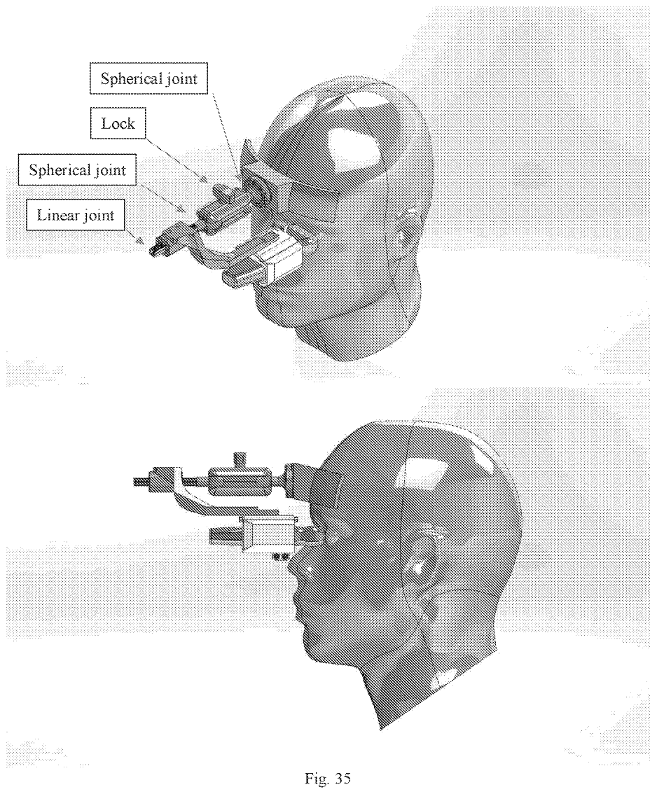

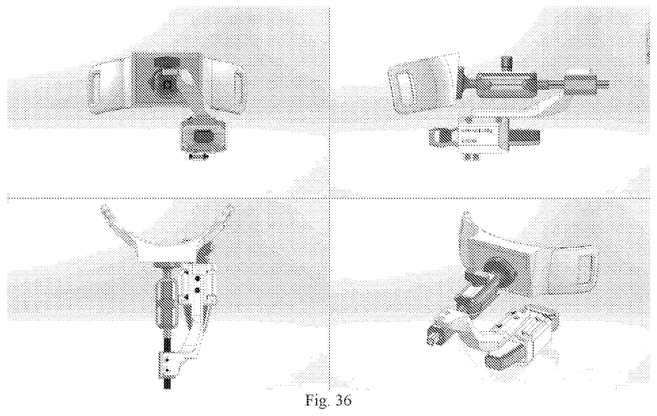

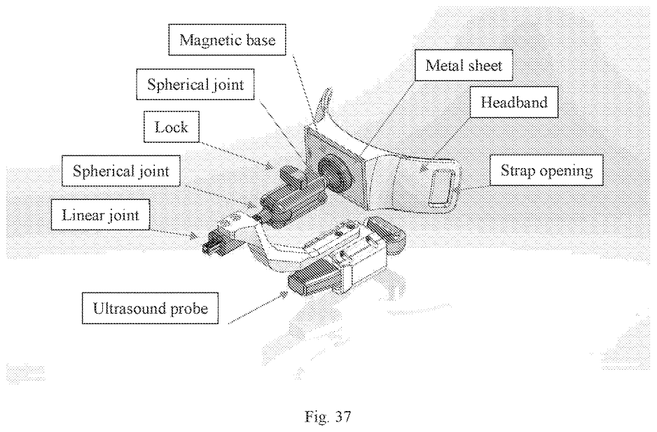

1) A novel ultrasound stabilization wearable to enable continuous monitoring and improve ultrasound image quality. Ocular ultrasound must be administered by a skilled clinician and takes several minutes for measurement of ONSD, which severely limits its use in busy NCCUs. We have developed the first wearable device designed specifically to enable stabilization of an ocular ultrasound probe toward continuous, non-invasive measurement of ICP. This enables safe monitoring over long-term timeframes and reduces inter-operator variability.
2) Autosegmentation and interpretation for real-time ICP estimation. Continuous, non-invasive monitoring re-quires a handsfree solution and automatic measurement and interpretation of ocular ultrasound images. We have developed an autosegmentation model that can accurately identify the optic nerve sheath and calculate the ONSD. Our model has demonstrated accuracy within 97% of ONSD as measured by a clinical expert.
3) Integration of ONSD+dynamic signals to improve ICP estimation accuracy. The stabilization devices disclosed herein enable, for the first time, the potential to integrate dynamic physiological signals that are associated with ICP into a continuous measurement platform. Several motion-based or dynamic parameters have been identified in ocular ultrasound that are correlated with ICP including the deformability index, heart rate, and motions associated with the ocular nerve and surrounding tissue. However, noise in typical handheld ultrasound often obscures these signals, eliminating the potential for use in ICP estimation. We have, furthermore, identified additional high frequency signals that have not previously been reported in the ocular ultrasound literature that are likely associated with elastomechanical properties of tissue adjacent to the optical nerve and that are likely correlated with ICP (see FIGS. 18 and 34). However, there have been reports of similar dynamic frequencies, in the range of 5-30 Hz, in other brain tissue and with different sensing modalities including invasive monitoring and acoustic cranial signals.^{11-13,37,77,78} Integration of dynamic signals present in ocular ultrasound holds significant potential to deliver the first non-invasive approach for accurate estimation of ICP, which would transform ICP monitoring.

There is a substantial interest in non-invasive methods, where the market share for non-invasive methods is expected to grow at 8.8% CAGR through 2030.⁸⁵ Furthermore, invasive ICP monitoring is only administered for patients at high risk of elevated ICP (i.e., moderate to severe TBI, acute ischemic stroke, and others). There is significant potential for increased monitoring of mild and moderate risk cases that are not candidates for invasive ICP monitoring, who currently receive little to no ICP monitoring. Embodiments of the invention can include can deliver the first non-invasive, continuous monitoring approach that can deliver quantitative estimation of ICP. This can enable more patients to receive the ICP monitoring that they need and may reduce the use of invasive ICP monitoring. Ultrasound imaging has previously been approved by the FDA for use in ocular sonography used for screening of elevated ICP.

We have developed illustrative embodiments of wearable systems that enable continuous, hands-free ocular ultrasound monitoring (FIG. 33), with a third prototype under development. The version 2 prototype (see FIG. 33) has been used for all following data collection. The device is designed to accurately position and consistently hold an ocular ultrasound probe securely at the patient's upper eyelid. The stabilizing headframe also securely holds the ultrasound probe across different body positions, including upright, supine, and the Trendelenburg positions that may occur in the NCCU. Ultrasound recordings are currently recorded in 15-second video files that are taken of the patient's bilateral eyes and optic nerves, with a temporal resolution of 30 frames per second. We are currently advancing development of a version 3 prototype that improves the adjustability, ease, and accuracy of locking in the probe through design ergonomics and higher quality components. Furthermore, the version 3 design can contain the electronics from a deconstructed ultrasound probe embedded into a low-profile, 3D printed custom housing for consistent and safe application to the upper eyelid. This enables a safe, hands-free approach for continuous insonation, where data can be collected and fed into our ML-pipeline. Iterative device development is ongoing and can be conducted in parallel with this project.

Autosegmentation ML algorithms can accurately resolve the optical nerve sheath diameter. A critical task in enabling true continuous, non-invasive monitoring is automatically segmenting and interpreting longitudinal ultra-sound images collected from the wearable to deliver ICP measurements on a convenient bed-side monitor. We have developed an autosegmentation algorithm that automates the identification and measurement of the optic nerve sheath. To perform autosegmentation of the optic nerve sheath, we have used a modified U-Net model provided by Tensorflow, which is a novel deep learning tool based on a convolutional neural network that has been developed for use in biomedical image segmentation. We created a data pipeline to interface the U-Net model to accept ocular ultrasound images and labeled binary masks of the optical nerve sheath as inputs. We demonstrated successful segmentation using a labeled dataset of 3,600 images from a study subject that were labeled using FIJI. Using reserved test images, we demonstrated that the model was able to identify the segmented region of interest (ROI) associated with the optic nerve sheath with 96% accuracy.

We then conducted a study to assess the capabilities for segmenting and measuring the ONSD in healthy participants. A total of 128 discreet images were used from 12 participants, where data from 9 participants were used for training and data from the remaining 3 participants were used for testing (75/25 split for training and testing). We implemented data augmentation such as rotating and flipping images to increase the effective dataset by approximately 10× for this proof-concept. All images were examined by an expert to determine the ONSD measurement and compare, where a clinical cutoff of 5.6 mm as determined from ocular ultrasound is used to indicate elevated ICP. Based on this analysis of approximately 1000 images, we demonstrated that the U-Net algorithm calculated the ONSD within nearly 97% of the expert measurement (FIG. 22). This demonstrates proof-of-concept for automatic measurement of the ONSD towards the continuous, non-invasive measurement of ICP. One of the primary goals of embodiments of the invention is to further optimize and validate the algorithm in the target patient population, i.e., TBI and stroke patients at risk of elevated ICP that are admitted to the NCCU.

Ultrasound stabilization enables resolution of additional physiological signals toward improving ICP estimation accuracy. Ocular ultrasound and ONSD measurement are currently used as a binary qualitative measurement of elevated ICP. However, there is significant potential to improve ICP estimation with ocular ultrasound through advanced data processing methods and introduction of additional supportive physiological signals that are correlated with ICP. For example, a parameter termed the deformability index, which measures the lateral motion of the optic nerve as it varies with heart rate, has been suggested to improve ICP estimation as the deformability is inversely related to stiffness, which increases with ICP. While several physiological signals could be used to improve estimation, only ONSD has been used clinically to determine elevated ICP, as it can be measured by a clinician at the bedside. However, innovative advanced dynamic parameters that are correlated with ICP, such as the de-formability index and brain tissue dynamics, require advanced signal processing algorithms. These parameters are also highly susceptible to motion-related arti-facts and excessive noise that result from operator-based ultrasound procedures. We have demonstrated that the use of a stabilizing device during ultra-sound imaging can significantly improve image quality and facilitate the extraction of physiological signals that can improve ICP correlation. FIG. 18 shows a typical ultrasound frame selected from a video compared to those that were motion-corrected and those that were collected with the stabilization device. Analysis of the collected ultrasound video from embodiments of the invention in the frequency domain enables identification of discrete physiological signals that are lost to noise in ultrasound videos collected by hand. We demonstrated successful resolution of both heart rate and high frequency motions that can be extracted from a sequence of ultrasound images. Heart rate can be resolved from pixel motion. Using images collected from the healthy UCLA participant cohort and heart rate measurements, we extracted the average heart rate from the ultrasound images and compared to the subject's heart rate that was recorded after each 15-second ultrasound image series acquisition and successfully calculated heart rate with a mean error (standard deviation) of −0.46 (3.67) beats/min compared to free-hand error of 9.46 (13.02) beats/min (FIG. 21). As the deformability index has been used to improve correlation with ICP and it is associated with cardiac cycle motion, this provides evidence that the stabilization device and ML approach can improve our ICP estimation performance.

In our studies we identified three additional high frequency motions in the ultrasound image sequences (see three additional labeled peaks in FIG. 18) as HF1-7.5 Hz, HF2-9.5 Hz, and HF3-14 Hz. To the best of our knowledge, we have not identified previous reports of similar high frequency motions with ocular ultrasound in the literature. We expect that these high frequency components are associated with elastomechanical properties of tissue and may further contribute to a correlation with ICP. FIG. 34 shows heart rate data derived from ultrasound and heart rate monitor, in addition to high frequency signals. Data was collected in both the upright and supine positions, or 0° and 90°, respectively. FIG. 34 bottom panel shows that when subjects change between supine and upright positions HF1 generally decreases and HF2 and HF3 increase (for all participants except S09, which could be due to a data collection error or S09 may be an outlier). Studies have demonstrated that ICP changes across different postural positions and further that ICP change across different postural position could be used to distinguish healthy patient groups from hydrocephalus and idiopathic intracranial hypertension.86 HF signal changes across supine and upright positions are likely to be more significant in neurocritical care patients that are less able to regulate ICP. Furthermore, baseline variations exist across participants, and HF2 and HF3 generally increase in those subjects with higher HR, while HF1 decreases (e.g., see S06 and S11 in FIG. 34).

These results provide evidence that HF signals derived from ultrasound are correlated with ICP. Currently, these parameters require manual selection of an ROI, however, the autosegmentation algorithm that can be optimized and validated in this work can enable automation of ROI selection and hence extraction of HF signals. This can also support the integration of additional parameters. That may improve ICP accuracy such as the deformability index, heart rate, motions associated with the optic nerve, and demographics.

In our studies we have demonstrated proof-of-concept for both the stabilization wearable and the autosegmentation algorithm. These features enable continuous, non-invasive monitoring. Calculation of ONSD with our algorithm currently demonstrates excellent agreement with expert opinion. As ONSD is the only accepted physiological signal used to determine elevated ICP clinically, our approach currently meets the performance necessary for clinical use. In this project, we can further validate this through training and validation with neurocritical care patients that have elevated ICP. Critically, we can also collect and test temporal data to extract several physiological signals that have significant potential to improve ICP estimation, which may enable the first accurate quantitative estimation of ICP through ultrasound imaging.

We have demonstrated improved image stabilization and image quality using the device embodiments disclosed herein as compared to handheld ultrasound in healthy participants. However, ONSD is tightly regulated in healthy patients. We can demonstrate improved image quality in the target population, neurocritical care patients that are at-risk for elevated ICP, which includes more variation in ONSD and optical nerve sheath anatomy than healthy participants. We can collect a critical dataset that is necessary for optimization and validation of the autosegmentation algorithm and ML-based ICP estimation approach.

Ultrasound images can be collected with device embodiments of the invention and handheld devices, using the same probe, and image quality can be compared. A total of around 78 adult participants that have been admitted to the NCCU from the emergency department or have been transferred from outside hospitals can be included. Participants can include those with moderate to severe TBIs and subarachnoid hemorrhage, intracerebral hemorrhage, or acute ischemic stroke.

For ICP estimation, invasive ICP monitoring is required for accurate model building and validation, as current ocular ultrasound approaches are limited to determining elevated ICP through a clinical cutoff. Previous studies successfully classified elevated ICP with sensitivity and specificity of 85-96% and 71-88%, respectively, and demonstrated correlations between r=0.32 and 0.895.19-23 As this is the first study in the target populations, we have chosen a low-moderate correlation of 0.4. Although it is very probable that our data can reveal higher correlation, this sample size gives us the confidence to be able to detect it. Thus, we have determined that a sample size of 62 is required based on 90% power and a significance level of 0.05 (5%). Furthermore, considering a 20% attrition, we can use a sample size of 78 (62/0.8). Additionally, a previous study used similar autosegmentation methods to segment the optical nerve sheath with an accuracy of 80% using 201 images from 50 subjects, which further supports our power study sample size. All participants can be selected from those that are undergoing transorbital ultrasound for screening to determine their clinical and neurological status. All participant screening and consent procedures can be carried out by a research coordinator according to standard policies. A nurse or research coordinator can screen participants to ensure that they meet inclusion and exclusion criteria (Table 3). Consent can be obtained from the patient's designated surrogate upon admission to the NCCU and consent can be obtained subsequently from the participant when possible.

Upon consent, all demographic and measurements in Table 4 can be completed prior to the ocular ultrasound procedure. For participants that require invasive monitoring through either intra-parenchymal or intra-ventricular catheter-based procedures, the initial pressure reading after placement can also be collected. Ultrasound images can be collected using both freehand measurement by a skilled operator and with the stabilization devices disclosed herein. Both measurements can be collected with the same ultrasound probe. A sterile dressing can be applied to each eye sequentially to allow for ultrasound imaging of the bilateral eyes. For both freehand and ultrasound measurements, the operator can steer the ocular ultrasound probe to the optimal view for measuring the ONSD in t planes, including the transverse and sagittal. For freehand measurements, the probe can be held in place and the eye can be insonated for 15 seconds, where imaging both the transverse and sagittal plane can result in 30 seconds of imaging per eye across two positions for a total of 60 seconds per eye. Bilateral eye imaging across both planes can be conducted with the participant positioned at 30° and in a supine position. This process can be repeated using a stabilization device as disclosed herein, where the probe can be placed in the device and the operator can steer the probe to the appropriate position before securing it with the device. After securing the device 15 seconds of imaging (1 video recording) can be completed per eye and between 4 and 8 total video recordings total per patient can be completed. ICP (collected through invasive monitoring), blood pressure, and heart rate data can also be recorded simultaneously with both invention and freehand ultrasound measurements.

To demonstrate an improvement in image quality with the stabilization devices disclosed herein, we can compare ONSD across all ultrasound images. The individual frames from each ultrasound video can be interpreted by an expert in the field. A nominal ONSD can be defined by the expert image interpreter for each video recording based on the freehand ultrasound measurement. ONSD can then be measured for each frame or every 5-50 frames depending on variation in the images (an expert may deem frames equal if minimal variation is observed). Image quality can be assessed by descriptive and inferential statistic including mean, variance, and hypothesis testing relative to the nominal diameter. We can demonstrate that the ONSD image quality is improved with devices as disclosed herein. We can also assess the relative time that the optic nerve sheath is in focus across the two recording methods. The ONSD as determined from each frame taken with the illustrative device and freehand can be compared to the nominal diameter. There can be up to 450 difference calculations per 15 second ultrasound video recording. The frames with <0.2 mm difference relative to the nominal diameter can be counted as within focus and this proportion out of 450 total differences can be used to determine the relative time in focus. Thus, this characterizes how many frames (and how long) the ONSD is accurately measured within the known true value. Finally, we can conduct hypothesis testing to determine if the mean diameter (from the series of frames) is significantly different than the nominal diameter. Maintaining adequate image quality and focus across the entire measurement period has implications for ease of clinician use and can guide future development, where loss of image focus may necessitate automated steering for long-term longitudinal measurement.

Sex-related differences in overall prevalence are well known in TBI, acute ischemic stroke, and other indications that may be at risk of elevated ICP, where, e.g., men are approximately 40% more likely to suffer a TBI than women87 and women account for 57% of all stroke deaths.88 However, results demonstrating sex-related differences associated with elevated ICP have been mixed. For example, some studies have demonstrated different response to injury across sexes, where female sex hormones may affect inflammation, edema, and oxidative stress following TBI.^{89,90} Furthermore, one study has found more frequent brain swelling and elevated ICP in women compared to men following injury.⁹¹ However, several studies have found no difference in ICP or intracerebral hemorrhage following moderate to severe TBIs.⁹²⁻⁹⁴ Therefore, as elevated ICP is associated with indications that occur more often in both men and women, and because sex related differences are inconclusive, we intend to enroll equal amounts of men and women in this study and can assess both sex-dependent and sex-independent contributions to ICP.

Embodiments of the invention optimize and validate ML algorithm for autosegmentation and interpretation of ultrasound images. We have demonstrated excellent agreement in ONSD that is measured using our ML autosegmentation algorithm compared to expert opinion in healthy participants. We can further validate the autosegmentation algorithm in patients with variable ONSDs and subsequent ICPs using data collected as discussed above. Furthermore, with optimization and larger training datasets, anticipate improved segmentation performance and ONSD measure of <0.2 mm compared to expert opinion. Successful autosegmentation and accurate measurement of ONSD in this patient population can strongly support the use of embodiments of the invention for automating ocular ultrasound for continuous, non-invasive measurement. Furthermore, due to the improved image quality and significant noise reductions associated with these stabilization devices, we can assess the capability for two different computational models (multivariable regression and deep learning) to quantitatively estimate ICP. We can compare a multivariable regression model that integrates the ONSD with additional dynamic parameters against a deep learning neural network. Several parameters such as heart rate, BMI, brain tissue dynamics, and deform-ability index have been used to improve ICP estimation. The deformability index measures the lateral motion of the optic nerve as it varies with heart rate, where it has been used to improve correlation with ICP. Deformability is inversely related to stiffness which increases with ICP.1 Furthermore, dynamic motions in the brain such as the pulse waveform of intracranial pressure and intracranial pulsatility have been correlated with ICP.^{11,70,74} Our stabilization device enables extraction of these dynamic features and motivates the optimization of a non-invasive, quantitative algorithm of ICP. We can optimize the regression algorithm, compare this with a neural network, and validate clinical performance.

We can first generate a database of ultrasound images that can support model training and validation. All ultrasound images can be de-identified and transferred to a secure cloud database. We can screen the data and discard data that is of low quality. Out-of-plane motion can degrade dynamic image quality. Images can be assessed by a blinded operator and graded on a scale of 0 to 2, where 0 includes minimal pixel shift, 1 includes perceivable pixel shift without loss of optic nerve sheath appearance, and 2 includes distinct pixel shift with some loss of optic nerve sheath appearance. Images with a grade of 2 can be discarded. Furthermore, redundant images can be removed. The ultrasound probe delivers high resolution imaging with 30 frames per second. For autosegmentation training and validation purposes, a significant amount of the data derived from each 15 second video with the device can be redundant due to the image stability, where we anticipate selection of 1 or 2 unique images per 15 second video. For ICP estimation each screened video can be considered a dynamic data point and used for time-based feature extraction. All images and videos in the database can then have the anatomy of the optic nerve, optic nerve sheath, and globe labeled by a clinical expert to support deep learning model training. The clinical expert can also measure additional anatomical parameters such as the width of the eyeball, optic disc elevation, the diameter of the optic nerve, and can associate additional parameters such as BMI, head circumference, and sex amongst others for ICP regression. We can reserve 20% of the data for testing and leave out data for validation. We can validate our optic nerve sheath autosegmentation algorithm through the Dice coefficient, where the Dice coefficient is a measure of image overlap and is calculated as the fraction of pixel overlap between the algorithm output and the binary, ground truth labeled image. We can ensure that Dice coefficient is greater than 0.85 in this dataset, which is a common benchmark for autosegmentation accuracy. We can complete the testing on the reserved dataset and the validation of the model on the left-out patient data. All statistical analysis can be completed using R, MatLab, and Stata.

We can then develop a multivariable regression model (using linear or nonlinear regression models) that utilizes our optical nerve sheath autosegmentation algorithm (for ONSD, eye width, and diameter measurements) and integrates parameters listed in Table 6 including the dynamic ONS motion frequency and amplitude identified. The multivariable regression model can be used for estimation of ICP and assessed with the Coefficient of Determination.

In parallel, supervised ML modeling techniques such as decision trees can be used to develop regression models.⁵⁴ We can also develop and compare a supervised deep learning algorithm using labeled input (optic nerve sheath) and labeled output data (true ICP) that we can use to predict ICP. We can develop a convolutional neural network using a recurrent neural network (RNN),96 a Combined deep CNN-LSTM,⁹⁷ or ResNet architectures.^{98,99} The input to the neural network may include im-ages as well as other patient data such as blood pressure, heart rate, head circumference, age, sex, height, weight, body mass index, ethnicity, as well as the ONSD and dynamic data.

For the different ICP estimation models, we can assess the Correlation Coefficient and the Bland Altman analysis and plot. We can also evaluate classification accuracy with multiple parameters found in Table 5 including area under the receiver operating characteristic curve (AUROC), sensitivity, and specificity.

We anticipate that models with multiple parameters including time-based features can outperform ONSD only parameter models. See Milestone 2.3 in Table 5 for all metrics that can be compared. Based on the performances of the ML and the multivariable models, we can identify the higher performing model and progress that one toward commercialization as our primary ICP estimation model.

We can validate ONSD autosegmentation algorithms using clinical data from the target population and will have demonstrated that ICP estimated from a multivariable regression or a neural network show improved correlation with ICP compared to the standard ONSD model. We can then choose the best performing model to advance towards commercialization. In the case that estimated ICP with either a deep learning model or a regression model does not provide the desired performance or requires additional data, we can explore the accuracy of a multi class approach. Namely, we can develop unique models for different ICP ranges. This approach may allow higher accuracy within each class (or ICP range). It may be beneficial to have patient baseline data for their healthy state. We can explore other anatomic measures as well ONSD further back (~10 mm) as baseline estimates to include in our models.

### EXAMPLE 1 REFERENCES

(1) Padayachy, L.; Brekken, R.; Fieggen, G.; Selbekk, T. Noninvasive Transorbital Assessment of the Optic Nerve Sheath in Children: Relationship Between Optic Nerve Sheath Diameter, Deformability Index, and Intracranial Pressure. Operative neurosurgery (Hagerstown, Md.) 2019, 16 (6), 726-733.
(2) Lee, S. H., Kim, H. S.; Yun, S. J. Optic Nerve Sheath Diameter Measurement for Predicting Raised Intracranial Pressure in Adult Patients with Severe Traumatic Brain Injury: A Meta-Analysis. Journal of Critical Care 2020, 56, 182-187.
(3) Koziarz, A.; Sne, N.; Kegel, F.; Nath, S.; Badhiwala, J. H.; Nassiri, F.; Mansouri, A.; Yang, K.; Zhou, Q.; Rice, T.; Faidi, S.; Passos, E.; Healey, A.; Banfield, L., Mensour, M.; Kirkpatrick, A. W.; Nassar, A.; Fehlings, M. G.; Hawryluk, G. W. J.; Almenawer, S. A. Bedside Optic Nerve Ultrasonography for Diagnosing Increased Intracranial Pressure. Ann Intern Med 2019, 171 (12), 896-905.
(4) Guo, Y.; Chen, Y.; Shen, C.; Fan, D.; Hu, X.; Duan, J.; Chen, Y. Optic Nerve Sheath Diameter and Optic Nerve Sheath Diameter/Eyeball Transverse Diameter Ratio in Prediction of Malignant Progression in Ischemic Stroke. Frontiers in Neurology 2022, 13.
(5) Li, C.; Wang, C.-C.; Meng, Y.; Fan, J.-Y.; Zhang, J.; Wang, L.-J. Ultrasonic Optic Nerve Sheath Diameter Could Improve the Prognosis of Acute Ischemic Stroke in the Intensive Care Unit. Frontiers in Pharmacology 2022, 13.
(6) Aletreby, W.; Alharthy, A.; Brindley, P. G.; Kutsogiannis, D. J.; Faqihi, F.; Alzayer, W.; Balhahmar, A.; Soliman, I.; Hamido, H.; Alqahtani, S. A.; Karakitsos, D.; Blaivas, M. Optic Nerve Sheath Diameter Ultrasound for Raised Intracranial Pressure. Journal of Ultrasound in Medicine 2022, 41 (3), 585-595.
(7) Du, J.; Deng, Y.; Li, H.; Qiao, S.; Yu, M.; Xu, Q.; Wang, C. Ratio of Optic Nerve Sheath Diameter to Eyeball Transverse Diameter by Ultrasound Can Predict Intracranial Hypertension in Traumatic Brain Injury Patients: A Prospective Study. Neurocritical Care 2020, 32 (2), 478-485.
(8) Youm, J. Y.; Lee, J. H.; Park, H. S. Comparison of Transorbital Ultrasound Measurements to Predict Intracranial Pressure in Brain-Injured Patients Requiring External Ventricular Drainage. Journal of neurosurgery 2021, 136 (1), 257-263.
(9) Pansell, J.; Bell, M.; Rudberg, P., Friman, O.; Cooray, C. Optic Nerve Sheath Diameter Measurement by Ultrasound: Evaluation of a Standardized Protocol. Journal of neuroimaging: official journal of the American Society of Neuroimaging 2022, 32 (1), 104-110.
(10) Lochner, P.; Czosnyka, M.; Naldi, A.; Lyros, E.; Pelosi, P.; Mathur, S.; Fassbender, K.; Robba, C. Optic Nerve Sheath Diameter: Present and Future Perspectives for Neurologists and Critical Care Physicians.
Neurological sciences: official journal of the Italian Neurological Society and of the Italian Society of Clinical Neurophysiology 2019, 40 (12), 2447-2457.
(11) Wagshul, M. E.; Eide, P. K., Madsen, J. R. The Pulsating Brain: A Review of Experimental and Clinical Studies of Intracranial Pulsatility. Fluids and Barriers of the CNS 2011, 8 (1), 1-23.
(12) Kim, D. J.; Kim, H.; Jeong, E. J.; Lee, H. J.; Czosnyka, M.; Son, Y.; Kim, B. J.; Czosnyka, Z. Spectral Analysis of Intracranial Pressure: Is It Helpful in the Assessment of Shunt Functioning in-Vivo? Clinical Neurology and Neurosurgery 2016, 142, 112-119
(13) Goto, T.; Furihata, K.; Hongo, K. Natural Resonance Frequency of the Brain Depends on Only Intracranial Pressure: Clinical Research. Scientific Reports 2020 10:1 2020, 10 (1), 1-10.
(14) Marketing Clearance of Diagnostic Ultrasound Systems and Transducers-Guidance for Industry and Food and Drug Administration Staff.
(15) Rosenbaum, B. P.; Vadera, S.; Kelly, M. L.; Kshettry, V. R.; Weil, R. J. Ventriculostomy: Frequency, Length of Stay and in-Hospital Mortality in the United States of America, 1988-2010. Journal of Clinical Neuroscience 2014, 21 (4), 623-632.
(16) Mahto, N.; Owodunni, O. P.; Okakpu, U.; Kazim, S. F.; Varela, S.; Varela, Y.; Garcia, J., Alunday, R.; Schmidt, M. H., Bowers, C. A. Postprocedural Complications of External Ventricular Drains: A MetaAnalysis Evaluating the Absolute Risk of Hemorrhages, Infections, and Revisions. World Neurosurgery 2023, 171, 41-64.
(17) Palasz, J., D'Antona, L.; Farrell, S.; Elborady, M. A.; Watkins, L. D.; Toma, A. K. External Ventricular Drain Management in Subarachnoid Haemorrhage: A Systematic Review and Meta-Analysis. Neurosurgical Review 2022, 45 (1), 365-373.
(18) Robba, C.; Graziano, F.; Rebora, P.; Elli, F.; Giussani, C.; Oddo, M.; Meyfroidt, G.; Helbok, R.; Taccone, F. S.; Prisco, L.; Vincent, J.-L.; Suarez, J. L; Stocchetti, N.; Citerio, G.; SYNAPSE-ICU Investigators. Intracranial Pressure Monitoring in Patients with Acute Brain Injury in the Intensive Care Unit (SYNAPSEICU): An International, Prospective Observational Cohort Study. Lancet Neurol 2021, 20 (7), 548-558.
(19) Dubourg, J.; Javouhey, E.; Geeraerts, T.; Messerer, M., Kassai, B. Ultrasonography of Optic Nerve Sheath Diameter for Detection of Raised Intracranial Pressure: A Systematic Review and Meta-Analysis. Intensive care medicine 2011, 37 (7), 1059-1068.
(20) Raffiz, M.; Abdullah, J. M. Optic Nerve Sheath Diameter Measurement: A Means of Detecting Raised ICP in Adult Traumatic and Non-Traumatic Neurosurgical Patients. The American journal of emergency medicine 2017, 35 (1), 150-153.
(21) Soliman, I.; Johnson, G. G. R. J.; Gillman, L. M.; Zeiler, F. A.; Faqihi, F.; Aletreby, W. T.; Balhamar, A.; Mahmood, N. N.; Mumtaz, S. A.; Alharthy, A.; Lazaridis, C.; Karakitsos, D. New Optic Nerve Sonography Quality Criteria in the Diagnostic Evaluation of Traumatic Brain Injury. Critical care research and practice 2018, 2018.
(22) Jeon, J. P.; Lee, S. U.; Kim, S. E.; Kang, S. H.; Yang, J. S., Choi, H. J.; Cho, Y. J.; Ban, S. P.; Byoun, H. S.; Kim, Y. S. Correlation of Optic Nerve Sheath Diameter with Directly Measured Intracranial Pressure in Korean Adults Using Bedside Ultrasonography. PLOS ONE 2017, 12 (9), e0183170.
(23) Wang, J.; Li, K.; Li, H.; Ji, C.; Wu, Z.; Chen, H.; Chen, B. Ultrasonographic Optic Nerve Sheath Diameter Correlation with ICP and Accuracy as a Tool for Noninvasive Surrogate ICP Measurement in Patients with Decompressive Craniotomy. Journal of neurosurgery 2019, 133 (2), 514-520.
(24) Launey, Y. ICP Management by Osmotherapy with Mannitol and Hypertonic Saline in ICU: Real-Time Effect on Optic Nerve Sheath Diameter Monitoring by Ultrasound. Neurosonology in Critical Care 2022, 1025-1036.
(25) Center for Disease Control and Prevention. National Center for Health Statistics: Mortality Data on CDC WONDER.
(26) Moderate and Severe TBI|Concussion|Traumatic Brain Injury|CDC Injury Center.
(27) Daugherty, J., Waltzman, D.; Sarmiento, K.; Xu, L. Traumatic Brain Injury-Related Deaths by Race/Ethnicity, Sex, Intent, and Mechanism of Injury-United States, 2000-2017. MMWR. Morbidity and Mortality Weekly Report 2019, 68 (46), 1050-1056.
(28) Carney, N.; Totten, A. M.; O'Reilly, C.; Ullman, J. S.; Hawryluk, G. W. J.; Bell, M. J.; Bratton, S. L.; Chesnut, R.; Harris, O. A.; Kissoon, N.; Rubiano, A. M.; Shutter, L.; Tasker, R. C.; Vavilala, M. S.; Wilberger, J.; Wright, D. W.; Ghajar, J. Guidelines for the Management of Severe Traumatic Brain Injury, Fourth Edition. Neurosurgery 2017, 80 (1), 6-15.
(29) Schizodimos, T.; Soulountsi, V.; Iasonidou, C.; Kapravelos, N. An Overview of Management of Intracranial Hypertension in the Intensive Care Unit. Journal of Anesthesia 2020, 34 (5), 741-757.
(30) Badri, S.; Chen, J.; Barber, J.; Temkin, N. R.; Dikmen, S. S.; Chesnut, R. M.; Deem, S.; Yanez, N. D.; Treggiari, M. M. Mortality and Long-Term Functional Outcome Associated with Intracranial Pressure after Traumatic Brain Injury. Intensive Care Medicine 2012, 38 (11), 1800-1809.
(31) Hansen, H.-C.; Helmke, S. S.; Helmke, K. Time Course of Optic Nerve Sheath Dilation: In Vitro Response Characteristics to Controlled Pressure Elevations. Journal of the Neurological Sciences 2022, 441, 120358.
(32) Luchette, M.; Helmke, K.; Maissan, I. M.; Hansen, H.-C.; Stolker, R. J.; Tasker, R. C.; Akhondi-Asl, A. Optic Nerve Sheath Viscoelastic Properties: Re-Examination of Biomechanical Behavior and Clinical Implications. Neurocrit Care 2022, 37 (1), 184-189.
(33) Hansen, H.-C.; Lagrèze, W.; Krueger, O.; Helmke, K. Dependence of the Optic Nerve Sheath Diameter on Acutely Applied Subarachnoidal Pressure—an Experimental Ultrasound Study. Acta Ophthalmol 2011, 89 (6), e528-532.
(34) Robba, C. Measuring Optic Nerve Sheath Diameter Using Ultrasonography for the Detection of Non Invasive Intracranial Pressure: What It Is and What It Is Not. Arq Neuropsiquiatr 2022, 80 (6), 547-549.
(35) Agrawal, D.; Raghavendran, K.; Zhao, L.; Rajajee, V. A Prospective Study of Optic Nerve Ultrasound for the Detection of Elevated Intracranial Pressure in Severe Traumatic Brain Injury. Crit Care Med 2020, 48 (12), e1278-e1285.
(36) Padayachy, L., Brekken, R., Fieggen, G., Selbekk, T. Pulsatile Dynamics of the Optic Nerve Sheath and Intracranial Pressure: An Exploratory In Vivo Investigation. Neurosurgery 2016, 79 (1), 100-107.
(37) Michaeli, D.; Rappaport, Z. H. Tissue Resonance Analysis; a Novel Method for Noninvasive Monitoring of Intracranial Pressure. Technical Note. J Neurosurg 2002, 96 (6), 1132-1137.
(38) Hirzallah, M. I.; Lochner, P.; Hafeez, M. U.; Lee, A. G.; Krogias, C.; Dongarwar, D., Manchanda, R.; Ouellette, L.; Hartman, N. D.; Ertl, M., Schlachetzki, F.; Robba, C. Quality Assessment of Optic Nerve Sheath Diameter Ultrasonography: Scoping Literature Review and Delphi Protocol. J Neuroimaging 2022, 32 (5), 808-824.
(39) Benhur, A.; Sharma, J.; Karna, S. T.; Shrivastava, A.; Saigal, S.; Waindeskar, V. V. Analysis of Dynamic Changes in Optic Nerve Sheath Diameter (ONSD) with Ultrasound in Post-Craniotomy Patients: Trends and Correlation with Computed Tomography ONSD and Glasgow Coma Scale in Post-Operative Period. J Neurosci Rural Pract 2022, 13 (4), 676-683.
(40) Bayramov, T.; Kilicaslan, B., Akinci, S. B.; Boyraz, G. The Effect of Pneumoperitoneum and Trendelenburg Position on Optic Nerve Sheath Diameter in Patients Undergoing Laparoscopic Hysterectomy. J Obstet Gynaecol Res 2022, 48 (3), 830-837.
(41) Mclaughlin, D.; Anderson, L.; Guo, J.; McNett, M. Serial Optic Nerve Sheath Diameter via Radiographic Imaging: Correlation With ICP and Outcomes. Neurol Clin Pract 2021, 11 (5), e620-e626.
(42) Relationship of the optic nerve sheath diameter and repeated invasive intracranial pressure measures in traumatic brain injury patients; a diagnostic accuracy study|Frontiers in Emergency Medicine.
(43) Biggs, A.; Lovett, M.; Moore-Clingenpeel, M.; O'Brien, N. Optic Nerve Sheath Diameter Does Not Correlate with Intracranial Pressure in Pediatric Neurocritical Care Patients. Childs Nerv Syst 2021, 37 (3), 951-957.
(44) Subramanian, S.; Nair, S.; Moorthy, R. K.; Rebekah, G., Krishnaprabhu, R.; Joseph, B. V.; Rajshekhar, V. Utility of Serial Optic Nerve Sheath Diameter Measurements in Patients Undergoing Cerebral Spinal Fluid Diversion Procedures for Hydrocephalus. World Neurosurgery 2021, 154, e168-e175.
(45) Vijay, P.; Lal, B. B., Sood, V.; Khanna, R.; Patidar, Y.; Alam, S. Dynamic Optic Nerve Sheath Diameter (ONSD) Guided Management of Raised Intracranial Pressure in Pediatric Acute Liver Failure. Hepatol Int 2021, 15 (2), 502-509.
(46) Bender, M.; Lakicevic, S.; Pravdic, N.; Schreiber, S.; Malojcic, B. Optic
Nerve Sheath Diameter Sonography during the Acute Stage of Intracerebral Hemorrhage: A Potential Role in Monitoring Neurocritical Patients. Ultrasound J 2020, 12 (1), 47.
(47) Naldi, A.; Pivetta, E.; Coppo, L.; Cantello, R.; Comi, C.; Stecco, A.; Cerrato, P.; Lesmeister, M.; Lochner, P. Ultrasonography Monitoring of Optic Nerve Sheath Diameter and Retinal Vessels in Patients with Cerebral Hemorrhage. J Neuroimaging 2019, 29 (3), 394-399.
(48) Beşir, A.; Tertemiz, O. F.; Akdoğan, A.; Duman, E. N. The Importance of Optic Nerve Sheath Diameter in Post-Dural Puncture Headache Diagnosis and Follow-Up. Noro Psikiyatr Ars 2019, 56 (3), 195-199.
(49) Thotakura, A. K., Marabathina, N. R.; Danaboyina, A. R.; Mareddy, R. R. Role of Serial Ultrasonic Optic Nerve Sheath Diameter Monitoring in Head Injury. Neurochirurgie 2017, 63 (6), 444-448.
(50) Klinzing, S.; Hilty, M. P.; Bechtel-Grosch, U.; Schuepbach, R. A.; Bühler, P.; Brandi, G. Dynamic Optic Nerve Sheath Diameter Changes upon Moderate Hyperventilation in Patients with Traumatic Brain Injury. J Crit Care 2020, 56, 229-235.
(51) Chopra, A.; Das, P. K.; Parashar, S., Misra, S.; Tripathi, M., Malviya, D.; Singh, D. Clinical Relevance of Transorbital Ultrasonographic Measurement of Optic Nerve Sheath Diameter (ONSD) for Estimation of Intracranial Pressure Following Cerebrospinal Fluid Diversion Surgery. Cureus 2022, 14 (5), e25200.
(52) Chen, L.-M.; Wang, L.-J.; Hu, Y., Jiang, X.-H.; Wang, Y.-Z.; Xing, Y.-Q. Ultrasonic Measurement of Optic Nerve Sheath Diameter: A Non-Invasive Surrogate Approach for Dynamic, Real-Time Evaluation of Intracranial Pressure. Br J Ophthalmol 2019, 103 (4), 437-441.
(53) Roldan, M.; Abay, T. Y.; Uff, C.; Kyriacou, P. A. A Pilot Clinical Study to Estimate Intracranial Pressure Utilising Cerebral Photoplethysmograms in Traumatic Brain Injury Patients. medRxiv May 24, 2023, p 2023.05.23.23290325.
(54) Abdul-Rahman, A.; Morgan, W.; Yu, D.-Y. A Machine Learning Approach in the Non-Invasive Prediction of Intracranial Pressure Using Modified Photoplethysmography. PLOS One 2022, 17 (9), e0275417.
(55) Tabassum, S.; Ruesch, A.; Acharya, D.; Yang, J.; Relander, F. A. J.; Scammon, B.; Wolf, M. S.; Rakkar, J.; Clark, R. S. B.; McDowell, M. M.; Kainerstorfer, J. M. Clinical Translation of Noninvasive Intracranial Pressure Sensing with Diffuse Correlation Spectroscopy. J Neurosurg 2023, 139 (1), 184-193.
(56) Miyagawa, T.; Sasaki, M.; Yamaura, A. Intracranial Pressure Based Decision Making: Prediction of Suspected Increased Intracranial Pressure with Machine Learning. PLOS One 2020, 15 (10), e0240845.
(57) Zhu, S.; Cheng, C., Zhao, D.; Zhao, Y.; Liu, X.; Zhang, J. The Clinical and Prognostic Values of Optic Nerve Sheath Diameter and Optic Nerve Sheath Diameter/Eyeball Transverse Diameter Ratio in Comatose Patients with Supratentorial Lesions. BMC Neurol 2021, 21, 259.
(58) Onder, H.; Goksungur, G.; Eliacik, S.; Ulusoy, E. K.; Arslan, G. The Significance of ONSD, ONSD/ETD Ratio, and Other Neuroimaging Parameters in Idiopathic Intracranial Hypertension. Neurol Res 2021, 43 (12), 1098-1106.
(59) Kim, D. H.; Jun, J.-S., Kim, R. Ultrasonographic Measurement of the Optic Nerve Sheath Diameter and Its Association with Eyeball Transverse Diameter in 585 Healthy Volunteers. Sci Rep 2017, 7 (1), 15906.
(60) Vaiman, M.; Sigal, T.; Kimiagar, I.; Bekerman, I. Noninvasive Assessment of the Intracranial Pressure in Non-Traumatic Intracranial Hemorrhage. J Clin Neurosci 2016, 34, 177-
(61) Yu, Z., Xing, Y.; Li, C.; Wang, S.; Song, X.; Wang, C.; Wang, L. Ultrasonic Optic Disc Height Combined with the Optic Nerve Sheath Diameter as a Promising Non-Invasive Marker of Elevated Intracranial Pressure. Frontiers in Physiology 2023, 14.
(62) Tessaro, M. O.; Friedman, N.; Al-Sani, F.; Gauthey, M.; Maguire, B.; Davis, A. Pediatric Point-of-Care Ultrasound of Optic Disc Elevation for Increased Intracranial Pressure: A Pilot Study. Am J Emerg Med 2021, 49, 18-23.
(63) Xu, G., Wu, X.; Yu, J.; Ding, H.; Ni, Z.; Wang, Y. Non-Invasive Intracranial Pressure Assessment Using Shear-Wave Elastography in Neuro-Critical Care Patients. Journal of Clinical Neuroscience 2022, 99, 261-267.
(64) Kreft, B.; Tzschätzsch, H.; Shahryari, M.; Haffner, P.; Braun, J.; Sack, I.; Streitberger, K.-J. Noninvasive Detection of Intracranial Hypertension by Novel Ultrasound Time-Harmonic Elastography. Invest Radiol 2022, 57 (2), 77-84.
(65) Razek, A. A. K. A.; Elsaid, N.; Belal, T., Batouty, N.; Azab, A. Combined Accuracy of Optic Nerve Sheath Diameter, Strain Ratio, and Shear Wave Elastography of the Optic Nerve in Patients with Idiopathic Intracranial Hypertension. Ultrasonography 2022, 41 (1), 106-113.
(66) Kaya, F. S., Bayram, E.; İnci, E. Evaluating the Optic Nerve Stiffness and Optic Nerve Sheath Diameter in Idiopathic Intracranial Hypertension Patients after the Resolution of Papilledema. Neurol Sci 2021, 42 (12), 5165-5170.
(67) Asal, N.; inal, M., Şahan, M. H.; Say, B. Assessment of the Optic Nerve Using Strain and Shear-Wave Elastography in Patients with Pseudotumour Cerebri. Clinical Radiology 2020, 75 (8), 629-635.
(68) Lee, H. C.; Lec, W. J.; Dho, Y. S.; Cho, W. S.; Kim, Y. H.; Park, H. P. Optic Nerve Sheath Diameter Based on Preoperative Brain Computed Tomography and Intracranial Pressure Are Positively Correlated in Adults with Hydrocephalus. Clinical neurology and neurosurgery 2018, 167, 31-35.
(69) Toscano, M.; Spadetta, G., Pulitano, P.; Rocco, M.; Di Piero, V.; Mecarelli, O.; Vicenzini, E. Optic Nerve Sheath Diameter Ultrasound Evaluation in Intensive Care Unit: Possible Role and Clinical Aspects in Neurological Critical Patients' Daily Monitoring. BioMed Research International 2017, 2017.
(70) Cardim, D.; Robba, C.; Bohdanowicz, M.; Donnelly, J.; Cabella, B., Liu, X.; Cabeleira, M.; Smielewski, P., Schmidt, B., Czosnyka, M. Non-Invasive Monitoring of Intracranial Pressure Using Transcranial Doppler Ultrasonography: Is It Possible? Neurocritical care 2016, 25 (3), 473-491.
(71) Weerakkody, R. A.; Czosnyka, M.; Zweifel, C.; Castellani, G.; Smielewski, P.; Brady, K.; Pickard, J. D.; Czosnyka, Z. Near Infrared Spectroscopy as Possible Non-Invasive Monitor of Slow Vasogenic ICP Waves. Acta neurochirurgica. Supplement 2012, 114, 181-185.
(72) Firsching, R.; Müller, C.; Pauli, S. U.; Voellger, B.; RöHL, F. W.; Behrens-Baumann, W. Noninvasive Assessment of Intracranial Pressure with Venous Ophthalmodynamometry. Clinical Article. Journal of neurosurgery 2011, 115 (2), 371-374.
(73) Firsching, R.; Schütze, M., Motschmann, M.; Behrens-Baumann, W. Venous Opthalmodynamometry: A Noninvasive Method for Assessment of Intracranial Pressure. Journal of neurosurgery 2000, 93 (1), 33-36.
(74) Querfurth, H. W.; Arms, S. W.; Lichy, C. M.; Irwin, W. T.; Steiner, T. Prediction of Intracranial Pressure from Noninvasive Transocular Venous and Arterial Hemodynamic Measurements: A Pilot Study. Neurocritical care 2004, 1 (2), 183-194.
(75) Lussier, B. L.; Olson, D. W. M.; Aiyagari, V. Automated Pupillometry in Neurocritical Care: Research and Practice. Current neurology and neuroscience reports 2019, 19 (10).
(76) McNett, M.; Moran, C.; Janki, C.; Gianakis, A. Correlations Between Hourly Pupillometer Readings and Intracranial Pressure Values. The Journal of neuroscience nursing: journal of the American Association of Neuroscience Nurses 2017, 49 (4), 229-234.
(77) Contant, C. F.; Robertson, C. S.; Crouch, J.; Gopinath, S. P.; Narayan, Raj. K.; Grossman, R. G. Intracranial Pressure Waveform Indices in Transient and Refractory Intracranial Hypertension. Journal of Neuroscience Methods 1995, 57 (1), 15-25.
(78) Bray, R. S.; Sherwood, A. M.; Halter, J. A.; Robertson, C.; Grossman, R. G. Development of a Clinical Monitoring System by Means of ICP Waveform Analysis. In Intracranial Pressure VI; Miller, J. D., Teasdale, G. M., Rowan, J. O., Galbraith, S. L., Mendelow, A. D., Eds.; Springer: Berlin, Heidelberg, 1986; pp 260-264.
(79) Purkayastha, S.; Sorond, F. Transcranial Doppler Ultrasound: Technique and Application. Seminars in neurology 2012, 32 (4), 411-420.
(80) Evensen, K. B.; Eide, P. K. Measuring Intracranial Pressure by Invasive, Less Invasive or Non-Invasive Means: Limitations and Avenues for Improvement. Fluids and barriers of the CNS 2020, 17 (1).
(81) Meiburger, K. M.; Naldi, A.; Lochner, P.; Marzola, F. Automatic Segmentation of the Optic Nerve in Transorbital Ultrasound Images Using a Deep Learning Approach. 2021 IEEE International Ultrasonics Symposium (IUS) 2021.
(82) Netteland, D. F.; Aarhus, M.; Smistad, E.; Sandset, E. C.; Padayachy, L.; Helseth, E.; Brekken, R. Noninvasive Intracranial Pressure Assessment by Optic Nerve Sheath Diameter: Automated Measurements as an Alternative to Clinician-Performed Measurements. Frontiers in Neurology 2023, 14.
(83) Ranjbarzadeh, R., Dorosti, S.; Jafarzadeh Ghoushchi, S.; Safavi, S.; Razmjooy, N.; Tataei Sarshar, N.; Anari, S.; Bendechache, M. Nerve Optic Segmentation in CT Images Using a Deep Learning Model and a Texture Descriptor. Complex and Intelligent Systems 2022, 8 (4), 3543-3557.
(84) Global Brain Monitoring Market Size and Growth Forecast Report.
(85) Intracranial Pressure Monitoring Devices Market Report, 2030.
(86) Andresen, M.; Hadi, A.; Petersen, L. G.; Juhler, M. Effect of Postural Changes on ICP in Healthy and Ill Subjects. Acta Neurochirurgica 2014, 157 (1), 109-113.
(87) Faul, M.; Coronado, V. Epidemiology of Traumatic Brain Injury. Handbook of Clinical Neurology 2015, 127, 3-13.
(88) M, H. Deaths: Leading Causes for 2019. National Vital Statistics Report2 2021, 70 (1)
(89) Villapol, S.; Loane, D. J.; Burns, M. P. Sexual Dimorphism in the Inflammatory Response to Traumatic Brain Injury. Glia 2017, 65 (9), 1423-1438.
(90) Günther, M.; Plantman, S.; Davidsson, J.; Angéria, M.; Mathiesen, T.; Risling, M. COX-2 Regulation and TUNEL-Positive Cell Death Differ between Genders in the Secondary Inflammatory Response Following Experimental Penetrating Focal Brain Injury in Rats. Acta Neurochirurgica 2015, 157 (4), 649-659.
(91) Farin, A.; Deutsch, R.; Biegon, A.; Marshall, L. F. Sex-Related Differences in Patients with Severe Head Injury: Greater Susceptibility to Brain Swelling in Female Patients 50 Years of Age and Younger. Journal of Neurosurgery 2003, 98 (1), 32-36.
(92) Kirkness, C. J.; Burr, R. L., Mitchell, P. H.; Newell, D. W. Is There a Sex Difference in the Course Following Traumatic Brain Injury?
(93) Czosnyka, M.; Radolovich, D.; Balestreri, M.; Lavinio, A.; Hutchinson, P.; Timofeev, I.; Smielewski, P.; Pickard, J. D. Gender-Related Differences in Intracranial Hypertension and Outcome after Traumatic Brain Injury. Acta Neurochirurgica, Supplementum 2008, No. 102, 25-28.
(94) Kraus, J.; Hsu, P.; Schaffer, K.; Vaca, F.; Ayers, K.; Kennedy, F.; Afifi, A. A. Preinjury Factors and 3-Month Outcomes Following Emergency Department Diagnosis of Mild Traumatic Brain Injury. Journal of Head Trauma Rehabilitation 2009, 24 (5), 344-354.
(95) Wang, Z. Deep Learning in Medical Ultrasound Image Segmentation: A Review. 2020.
(96) Eom, H.; Lee, D.; Han, S.; Sun Hariyani, Y.; Lim, Y.; Sohn, I.; Park, K.; Park, C. End-To-End Deep Learning Architecture for Continuous Blood Pressure Estimation Using Attention Mechanism. Sensors (Basel, Switzerland) 2020, 20 (8).
(97) Jeong, D. U.; Lim, K. M. Combined Deep CNN-LSTM Network-Based Multitasking Learning Architecture for Noninvasive Continuous Blood Pressure Estimation Using Difference in ECG-PPG Features. Scientific Reports 2021 11:1 2021, 11 (1), 1-8.
(98) Alzubaidi, L.; Zhang, J.; Humaidi, A. J.; Al-Dujaili, A.; Duan, Y.; Al-Shamma, O.; Santamaría, J.; Fadhel, M. A.; Al-Amidie, M.; Farhan, L. Review of Deep Learning: Concepts, CNN Architectures, Challenges, Applications, Future Directions. Journal of Big Data 2021 8:1 2021, 8 (1), 1-74.
(99) Paviglianiti, A.; Randazzo, V.; Villata, S.; Cirrincione, G.; Pasero, E. A Comparison of Deep Learning Techniques for Arterial Blood Pressure Prediction. Cognitive Computation 2022, 14 (5), 1689-1710.

### EXAMPLE 2: ILLUSTRATIVE EMBODIMENT OF THE INVENTION

In another illustrative embodiment of the invention, the probe can be installed and can be held in place with the headframe. The eye can be insonated continuously for 15 seconds or longer, imaging the transverse, sagittal and as well as various intermediary planes. For example, considering just two planes, the probe can insonate continuously for 15 seconds in one the transverse plane, then switch to the and sagittal plane to insonate for an additional 15 seconds and repeat back in the transverse plane.

The insonation and recording process can either continue without interruption or the probe can stop insonating for a certain period before restarting and recording another set. This can be done in one eye only, in both eyes sequentially, or in both eyes simultaneously. The insonation can capture images at a frame rate of at least 30 Hz, with higher frame rates allowing higher frequency content to be resolved as per Nyquist theorem. The probe spatial resolution should be approximately 13.2 pixels/mm or better, with higher resolution allowing to resolve anatomical changes more accurately in the images.

The periods between insonations can be determined based on the patient's status in order not to miss important clinical events. The clinician can be able to program the device to a specific interval of insonation or set it automatically where the device can determine the interval based on the intracranial pressure (ICP) assessment. Additionally, during the periods between insonations, the device may automatically release physical contact between the probe and the eye and resume the contact before the next insonation session begins.

### Segmentation

To assess changes in the structures of the eye, the optic nerve, and the optic nerve sheath anatomy we first need to identify and segment these anatomies of interest. Once segmented, anatomical information can be measured describing their shape, change in shape, as well as the rate of change.

Image segmentation software can be based on artificial intelligence such as a machine learning (ML) programs where a computer software is designed to learn to identify patterns in the shape and structure of an anatomy [1-2]. Several non-ML based methods [3-6] have also been developed specifically for segmentation of the structures in the back of the eye. These methods are designed to leverage humans' prior knowledge of the patterns in the anatomy.

### Image Processing

The key utility of segmentation is that it facilitates detection of changes in a specific anatomy which can reflect changes in disease state. Some measurements or parameters can be extracted directly from discrete or static images (e.g., diameter of the optic nerve). Other parameters can only be extracted by implementing mathematical operations that enable their identification wither because they are too subtle for the human eye, they represent patterns that the human brain isn't adept at distinguishing, or because of existing noise in the signal that prevents identification of the motion (e.g., motions of the optic nerve sheath). To quantify the changes between two frames in a sequence of frames of a video, we may look at time or frequency domain parameters. Since a frame or image is a collection of pixels, any measurement or operation is performed on pixels.

Spatial domain operations we may apply include operations to measure a pixel's position or a group of pixels' position and orientation in space such as block or feature matching image registration methods or methods to modify and enhance an image such as image filtering accomplished through convolution, which may help to identify:

Translation or rotation amplitude/distance of a particular pixel or a group of pixels
Direction of translation or rotation of a particular pixel or a group of pixels
Rate of change of translation or rotation of a particular pixel or a group of pixels
Shade or intensity of a pixel or a group of pixels
Rate of change of intensity of a particular pixel or a group of pixels
Certain features in the image by emphasizing features or removing other features
The mean, variance, maximum, and minimum of any of the above motion metrics
The above metrics may be measured between two pixels, two groups of pixels or between a pixel or a group of pixels relative to an inertial frame of reference

Spectral domain operations we may apply include Fourier series, Fourier transform, and Wavelet transform which may help to identify frequency characteristics in the image such as:
Frequency of motions of a particular pixel or a group of pixels
Frequency of intensity of a particular pixel or a group of pixels
Variations in frequencies of a particular pixel or a group of pixels over time

We may also apply image registration in the spectral domain such as phase-correlation or filtering such as a Finite Impulse Response (FIR) filter to modify the frequency content of images.

### Relationship to ICP

The image processing step may result in a variety of parameters that are associated with the anatomy of interest. These parameters may reflect the status of the disease (i.e., ICP). To evaluate the relationship between said parameters and ICP, several mathematical operations may be employed. In the case that there are multiple independent variables that are considered for estimation of ICP, multiple regression, which is an extension of simple linear regression, may be used and may have the following form:

$$\\ {y}_{i} = {β}_{0} + {β}_{1} {x}_{i 1} + {β}_{2} {x}_{i 2} + … + {β}_{p} {x}_{ip} + ε$$

where, for i=n observations:
yᵢ=dependent variable
xᵢ=explanatory/independent variables
β₀=y-intercept (constant term)
βₚ=slope coefficients for each explanatory variable
ε=the model's error term (also known as the residuals)

If a linear regression does not provide a good fit to the data, a nonlinear regression approach may be explored such as a rational function (ratio of two polynomial function) or a logarithmic function.

Alternatively, we may develop a deep learning neural network to predict the ICP (output). The input to the neural network may include images as well as other patient data such as blood pressure, heart rate, head circumference, age, sex, height, weight, body mass index, and ethnicity.

Certain embodiments of the invention can incorporate known elements or method steps used in this field of technology. For example, Nisonic (ocular ultrasound) makes a hand-held transducer optimized for ocular ultrasound (Nisonic P-100) for the detection of increased ICP. Machine learning software assists in image acquisition. CE. While optimized for ocular ultrasound, Nisonic uses a handheld device and therefore require an operator to be available to frequently perform the test. Nisonic assists with image acquisition but still relies on an operator to scan and find the correct plane, which may still result in a suboptimal image. In contrast, embodiments of the invention disclosed herein automatically scans to acquire the optimal image.
Several indications can affect the intracranial pressure, including head trauma, stroke, meningitis and other central nervous system infections, brain tumors. When brain pressure increases, the optic nerve sheath widens and becomes stiffer.
Nisonic combines the optic nerve sheath diameter (ONSD) with additional information gathered from the nerve sheath to estimate the pressure.
Has an AI algorithm to automatically identify the optic nerve but requires an operator to be available to frequently perform the test or clinically important events may be missed. Therefore, may collect sparse, static data points depending on the availability of the operator.
Requires an operator to position the probe in the right plane in order to acquire an image of the optic nerve.

Nisonic has also developed a method for analyzing ONS dynamics from transorbital ultrasound to differentiate between patients with raised ICP vs normal ICP [7]. WO2016193168 discloses a method for detecting the pulsatile dynamics of the optic nerve sheath, ONS that can be adapted/modified in embodiments of the invention. It involves steps to locate the optic nerve sheath, ONS, choosing one or more locations in the ONS or in the region surrounding the ONS, and measuring the pulsatile dynamic or displacement at said location.

In one embodiment, the method for detecting pulsatile dynamics comprises the step of performing a Fourier analysis of the motion pattern in any given direction.

In another embodiment, the method for detecting pulsatile dynamics comprises the step of obtaining the pulsatile dynamics by detecting displacement at two locations around the optic nerve sheath or in the region surrounding the and obtaining a parameter of deformability (Δ). The parameter of deformability may be calculated according to the equation (1):

$$\\ Δ = \frac{\vert {d}_{A} - {d}_{B} \vert}{{d}_{A} + {d}_{B}}$$

wherein (d_{A}) and (d_{B}) represents the displacement at each location around the ONS.

### Embodiment Differentiation

The general hypothesis is that ONS becomes stiffer as ICP rises (a simple analogy is a garden hose that becomes stiffer with increasing water pressure). This can result in motion amplitude variability of the sides of the ONS as demonstrated by Padayachy et al. Furthermore, Padayachy et al. extracted the motion amplitude component corresponding to the fundamental heart rate frequency.

In contrast, in embodiments of the invention, the approach is to demonstrate that stiffness variations in the ONS result in motion frequency variation. Because our pixel intensity method is more sensitive to anatomical motions compared to image registration methods as used by padayachy et al., we were able to detect not only motions associated with the heart rate cycle but also higher frequency motions that are presumed to be associated with brain tissue dynamics. There are several publications that support such brain tissue dynamics besides Padayachy's [8-13]

### EXAMPLE 2 REFERENCES

1. Meiburger, Kristen M., et al. “Automatic segmentation of the optic nerve in transorbital ultrasound images using a deep learning approach.” 2021 IEEE International Ultrasonics Symposium (IUS). IEEE, 2021.
2. Ranjbarzadeh, Ramin, et al. “Nerve optic segmentation in CT images using a deep learning model and a texture descriptor.” Complex & Intelligent Systems 8.4 (2022): 3543-3557.
3. Gerber, Samuel, et al. “Automatic estimation of the optic nerve sheath diameter from ultrasound images.” Imaging for Patient-Customized Simulations and Systems for Point-of-Care Ultrasound: International Workshops, BIVPCS 2017 and POCUS 2017, Held in Conjunction with MICCAI 2017, Québec City, QC, Canada, Sep. 14, 2017, Proceedings. Springer International Publishing, 2017.
4. Soroushmehr, Reza, et al. “Automated optic nerve sheath diameter measurement using super-pixel analysis.” 2019 41st Annual International Conference of the IEEE Engineering in Medicine and Biology Society (EMBC). IEEE, 2019.
5. Meiburger, Kristen M., et al. “Automatic optic nerve measurement: a new tool to standardize optic nerve assessment in ultrasound B-mode images.” Ultrasound in medicine & biology 46.6 (2020): 1533-1544.
6. Rajajee, Venkatakrishna, et al. “Novel algorithm for automated optic nerve sheath diameter measurement using clustering approach.” Military a Medicine 186.Supplement_1 (2021): 496-501.
7. Padayachy, Llewellyn, et al. “Pulsatile dynamics of the optic nerve sheath and intracranial pressure: an exploratory in vivo investigation.” Neurosurgery 79.1 (2016): 100-107.
8. Wagshul, Mark E., Per K. Eide, and Joseph R. Madsen. “The pulsating brain: a review of experimental and clinical studies of intracranial pulsatility. Fluids and Barriers of the CNS.” (2011).
9. Michaeli, David, and Z. Harry Rappaport. “Tissue resonance analysis: a novel method for noninvasive monitoring of intracranial pressure.” Journal of neurosurgery 96.6 (2002): 1132-1137.
10. Robertson, Claudia S., et al. “Clinical experience with a continuous monitor of intracranial compliance.” Journal of neurosurgery 71.5 (1989): 673-680.
11. Kim, Dong-Joo, et al. “Spectral analysis of intracranial pressure: Is it helpful in the assessment of shunt functioning in-vivo?.” Clinical Neurology and Neurosurgery 142 (2016): 112-119.
12. Goto, Tetsuya, Kenji Furihata, and Kazuhiro Hongo. “Natural resonance frequency of the brain depends on only intracranial pressure: Clinical research.” Scientific Reports 10.1 (2020): 1-13. Padayachy, Llewellyn, et al. “Noninvasive transorbital assessment of the optic nerve sheath in children: relationship between optic nerve sheath diameter, deformability index, and intracranial pressure.” Operative Neurosurgery 16.6 (2019): 726-733.

TABLES
Table 1. Non-invasive ICP monitoring competition

| | | Handheld | Transcranial | Near-infrared | Dynamometry | |
|---|---|---|---|---|---|---|
| | | ocular ultra- | Doppler Ultra- | sensors | (Third | Quantitative |
| | | sound | sound | (Cranio-) | Eye | Pupillometry |
| | CranioView | (Nisonic) | (Nova Signal) | Sense) | Diagnostics | (NeurOptics) |
| Continuous | Yes | No | Yes | Yes | No | No |
| monitoring | | | | | | |
| ICP correlation (r) | >ocular ultrasound | 0.32- | 0.36-0.8⁷⁰ | 0.55⁷¹ | 0.662-0.98⁷²⁻⁷⁴ | 0.28-0.55^{75, 76} |
| | (with dynamic parameters) | 0.895^{1, 18, 23, 58, 89} | | | | |
| Ease of use* | High | Moderate | Low | High | Low | High |
| Workflow* | High | Low | Low | Moderate | Low | Moderate |
| Clinical Adoption | High | High | Moderate | High | Moderate | Low |
| Reimbursement | High | High | Moderate | Low | Low | Low |
| potential | | | | | | |

*Ease of use refers to difficulty associated with measurement by a clinician, workflow refers to how compatible the device is with other clinical procedures and processes

TABLE 2
Success Criteria for Phase II - Aim 1

| Milestones | | Evaluation Method | Qualitative/Quantitative Metric |
|---|---|---|---|
| 1.1 | Recruit and enroll | Screen and consent participants | 100% enrollment |
| | patients | | |
| 1.2 | Collect study data | Patient history, ultrasound, invasive | Collect ultrasound images and ICP monitoring data from at |
| | | ICP monitoring, additional | least 74 participants |
| | | physiological signals | |
| 1.3 | Comparison of CranioView | Image analysis | ONSD mean difference (CranioView) < ONSD mean difference |
| | with freehand | | (freehand) |
| | ultrasound image | | ONSD Variance (CranioView) < ONSD Variance (freehand) |
| | quality | | Percent time image is focused defined as (number of frames |
| | | | with < 0.2 mm difference)/(total number of frames) with |
| | | | CranioView > freehand |

TABLE 3
Inclusion and exclusion criteria.
Inclusion criteria
18 years or older
Moderate to severe TBI (Glasgow Coma Scale score
3-12)
Subarachnoid hemorrhage
Intracerebral hemorrhage
Acute ischemic stroke
Patient requires intraparenchymal sensor or
intraventricular monitor
Exclusion criteria
Patients with facial or cranial trauma that present a
contraindication for continuous application of any
device to the head as determined by the treating
clinician
Any ophthalmologic or neuro-ophthalmologic
condition for which ultrasound imaging is precluded or
determined to be unsafe by the treating clinician or an
ophthalmologist
Prior ophthalmologic or neuro-ophthalmologic
surgery as determined by the treating clinician or an
ophthalmologist

TABLE 4
Study data

| Data category | Data to collected |
|---|---|
| Baseline data | ICP (through invasive monitoring), blood pressure, |
| | heart rate, head circumference, age, sex, height, |
| | weight, body mass index, and ethnicity |
| Ultrasound imaging (imaging | Between 4 and 8 15-second video recordings per |
| conducted with participant | patient will be recorded, where 15 seconds transverse |
| in 30° and supine | plane imaging or 15 seconds of sagittal plane |
| positions) | imaging per eye will be considered one video |
| | recording. Recordings will be conducted with the |
| | CranioView stabilization device and freehand |
| Collected simultaneously to | ICP, blood pressure, heart rate |
| ultrasound imaging | |

TABLE 5
Success Criteria for Phase II - Aim 2

| Milestones | | Evaluation Method | Qualitative/Quantitative Metric |
|---|---|---|---|
| 2.1 | Generate ultrasound | Database | Curated dataset with discrete labeled ultrasound images that are |
| | imaging database | development | associated with ICP from at least 78 participants |
| 2.2 | Refine and validate optic | Autosegmentation | Dice coefficient ≥ 0.85 |
| | nerve sheath and globe | validation | Other pixel evaluation indicators will be considered (e.g., recall, pixel |
| | autosegmentation | | accuracy and intersection over union)⁹⁵ |
| | algorithm | | \|ONSD (ML) − ONSD (expert)\| ≤ 0.2 mm |
| 2.3 | Develop regression and | ICP estimation with | Correlation: dynamic parameters + ONSD or deep learning model ≥ ONSD |
| | deep learning neural network | multivariable regression | Bland Altman bias and 95% range: dynamic parameters + ONSD ≤ ONSD |
| | algorithm for | and deep learning | Sensitivity: dynamic parameters + ONSD or deep learning model ≥ ONSD |
| | estimating ICP | model | Specificity: dynamic parameters + ONSD or deep learning model ≥ ONSD |
| | | | AUROC: dynamic parameters + ONSD or deep learning model ≥ ONSD |
| 2.4 | Compare ICP estimation | ICP estimation | Determine model that delivers better ICP estimation based on correlation |
| | with regression and | method comparison | and Bland Altman analysis |
| | deep learning models | | Determine model that delivers greatest Sensitivity, Specificity, and |
| | | | AUROC |

*ICP ≥ 20 mmHg will be used as a positive cut-off for all sensitivity and specificity calculations

TABLE 6
Multivariable regression algorithm variables

| | |
|---|---|
| ONSD | BMI, height, and weight |
| Deformability index | Cerebrovascular or |
| | cardiovascular disease |
| ONS motion frequency and | Diabetes |
| amplitude | |
| Pulse pressure | Hypertension |
| High frequency signals (HF₁- | Sex |
| 7.5 Hz, HF₂-9.5 Hz, and HF₃- | |
| 14 Hz), see preliminary data | |
| Eye width and diameter | Ethnicity |
| Head width and circumference | Smoking |
| Optic disc elevation (ODE) | |

TABLE 7
Project Timeline

| | | Quarter | | | | | | | |
|---|---|---|---|---|---|---|---|---|---|
| Task | Milestone | Q1 | Q2 | Q3 | Q4 | Q5 | Q6 | Q7 | Q8 |

| | |
|---|---|
| 1.1 | Recruit and enroll patients |
| 1.2 | Collect study data |
| 1.3 | Comparison of CranioView with freehand |
| | ultrasound and invasive monitoring |
| 2.1 | Generate ultrasound imaging database |
| 2.2 | Validate autosegmentation algorithm |
| 2.3 | Develop regression and deep learning |
| | models for ICP estimation |
| 2.4 | Compare ICP estimation for neural |
| | network and regression models |

Illustrative publications in this general field include U.S. patent publication and patent nos. US20130184584, US20150045012, US20140268037, US 20180132739, US20090306538, U.S. Pat. Nos. 7,618,372, 10,379,350, 9,398,861, 9,078,612, 9,585,578, 10,405,763, 7,670,002, 7,216,985, 6,116,736, 10,149,614, 7,967,442, 5,903,333, 6,637,881 and 6,773,407; and foreign patent publication nos. GB201908051, GB201908052, JP5274283, EP2977000, EP2567656 and EP2797494. All publications mentioned are incorporated herein by reference to disclose and describe aspects, methods and/or materials in connection with the cited publications.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention.

1. A system for estimating intracranial pressure in a subject comprising:
a headset adapted to rest on the head and/or face of the subject;
at least one sensing probe; wherein:
the sensing probe is coupled to the headset and adapted to capture a plurality of sensed parameters from a plurality of axes; wherein the system correlates one or more sensed parameters with intracranial pressure so as to estimate intracranial pressure in the subject noninvasively and optionally continuously.

2. The system of claim 1, wherein the headset comprises at least one of:
a longitudinal member adapted rest on an ear of the subject;
a longitudinal member adapted rest on the nose of the subject;
a supportive member adapted to rest on the cheek of the subject;
a flexible band adapted to secure the headset to the head of the subject; and
a deformable material embedded in the headset.

3. The system of claim 1, wherein the sensing probe(s) perform at least one process selected from:
an ultrasound process;
a shear-wave elastography process;
a pupillometry process;
a dynamometry process;
a near infrared spectroscopy process;
an acoustic tympanometry process;
an optical coherence tomography process; and
an electroencephalography process.

4. The system of claim 1 wherein the sensing probe(s) is coupled to a processor adapted to extract the sensed parameters made by the sensor probe, which change in concordance with changes in intracranial pressure.

5. The system of claim 1, wherein the processor controls the sensing probe position and orientation in a feedback loop, either through mechanical or electrical means, to optimize data acquisition.

6. The system of claim 1, wherein the sensed data is further analyzed by a separate software algorithm that automatically interprets and quantifies the described measurements, and is able to use these parameters to estimate intracranial pressure.

7. The system of claim 3 wherein the sensing probe:
comprises a composition adapted to conduct and receive ultrasonic waves;
is adapted to capture a plurality of images in multiple planes comprising at least one of:
an eyeball transverse diameter;
an eyeball anteroposterior diameter;
a concavity of an optic disc;
a convexity of an optic disc;
optic disc elevation;
an arclength of a globe;
a radius of a globe;
an optic nerve diameter;
an optic nerve sheath diameter;
a deformation, displacement or motion of an optic nerve; and
corresponding frequency spectra;
stiffness of the optic nerve and sheath;

8. The system of claim 1, wherein:
the sensing probe(s) performs an ultrasound process;
the system estimates intracranial pressure continuously; and
the system comprises a stabilization device that localizes the sensing probe to
a region of a patient's anatomy and locks the sensing probe in place at the region of the patient's anatomy.

9. The system of claim 8, wherein:
the stabilization device comprises one or more adjustable joints adapted to allow the sensing probe to move in a three-dimensional space;
the stabilization device locks the sensing probe to an eyelid of a patient; and/or
the system uses an autosegmentation algorithm to detect changes in the patient's anatomy.

10. A method of estimating intracranial pressure in a subject comprising:
coupling the system of any one of claim 1 on a subject; and
using the system to estimate intracranial pressure in a subject through an equation or set of equations based on the output from the automated software from claim 6 and demographic parameters of at least one of:
age;
gender;
height;
weight;
BMI;
head circumference;
head and brain width;
ethnicity; and
race;
or physiologic parameters of at least one of:
a body position;
a heart rate;
a respiratory rate;
a systolic blood pressure measurement;
a diastolic blood pressure measurement;
a mean arterial pressure measurement;
a cerebral perfusion pressure measurement;
an intracranial pressure measurement;
an intraocular pressure measurement;
a set of arterial blood gases measurements; and
an electrocardiogram tracing;
and/or comorbid conditions of at least one of:
diabetes;
hypertension;
cardiovascular disease;
cerebrovascular disease;
neurologic disorder;
ophthalmologic disorder; and
smoking status.

11. The method of claim 10, wherein the system performs an ultrasound process.

12. The method of claim 11, wherein the system estimates intracranial pressure continuously.

13. The method of claim 12, wherein the system comprises a stabilization device that localizes the sensing probe to a region of a patient's anatomy and locks the sensing probe in place at the region of the patient's anatomy.

14. The method of claim 13, wherein the stabilization device locks the sensing probe in place on an eyelid of a patient.

15. The method of claim 14, wherein the sensing probe(s) is coupled to a processor adapted to extract the sensed parameters, and the system uses an autosegmentation algorithm to detect changes in the patient's anatomy.
